(19) United States
(12) Patent Application Publication
(10) Pub. No.: US 2017/0044556 A1
(43) Pub. Date: Feb. 16, 2017

(54) STABILIZATION OF CYTOCHROME P450 REDUCTASE

(71) Applicant: DSM IP ASSETS B.V., Heerlen (NL)

(72) Inventors: Anita EMMERSTORFER, Graz (AT); Tamara WRIESSNEGGER, Graz (NL); Miriam WIMMER-TEUBENBACHER, Graz (AT); Harald PICHLER, Graz (AT); Monika MUELLER, Echt (NL); Iwona KALUZNA, Echt (NL); Martin SCHURMANN, Echt (NL)

(21) Appl. No.: 15/306,120

(22) PCT Filed: Apr. 28, 2015
(86) PCT No.: PCT/EP2015/059225

§ 371 Date: Oct. 24, 2016

(30) Foreign Application Priority Data
Apr. 25, 2014 (EP) 14165949.0

(51) Int. Cl. C12N 15/81; C12N 9/02; C07K 14/395; C12P 7/26

(52) U.S. Cl. CPC C12N 15/81; C12P 7/26; C12N 9/0073; C12N 9/0042; C12Y 114/13121; C12Y 106/02004; C07K 14/395

(57) ABSTRACT

The present invention relates to a recombinant microorganism which is capable of expressing a cytochrome P450 reductase (CPR) and, optionally, a cytochrome P450 enzyme (CYP) and which is capable of overexpressing a Ice2p comprising the amino acid sequence set out in SEQ ID NO: 2 or a sequence having at least 50% sequence identity thereto. The invention further relates to use of Ice2p to stabilize expression of a cytochrome P450 reductase in a recombinant microorganism.

### FIELD OF THE INVENTION

The present invention relates to a recombinant host cell in which the expression of a cytochrome P450 reductase enzyme is stabilized. The invention further relates to a method for the production of a compound of interest in a recombinant host cell and to a method for the production of a compound of interest in a biocatalytic reaction. The invention also relates to the use of a protein for stabilization of a cytochrome P450 reductase enzyme.

### BACKGROUND TO THE INVENTION

The cytochrome P450 superfamily of monooxygenases (officially abbreviated as CYP) is a large and diverse group of enzymes that catalyze the oxidation of organic substances. The substrates of CYP enzymes include metabolic intermediates such as lipids and steroidal hormones, as well as xenobiotic substances such as drugs and other toxic chemicals. CYPs are the major enzymes involved in drug metabolism and bioactivation, accounting for about 75% of the total number of different metabolic reactions.

CYP enzymes have been identified in all domains of life animals, plants, fungi, protists, bacteria, archaea and even in viruses. However, the enzymes have not been found in E. coli. More than 18,000 distinct CYP proteins are known.

Most CYPs require a protein partner to deliver one or more electrons to reduce the iron (and eventually molecular oxygen). Based on the nature of the electron transfer proteins CYPs can be classified into several groups. In microsomal P450 systems electrons are transferred from NADPH via cytochrome P450 reductase (variously CPR, POR, or CYPOR).

Given that cytochrome P450 reductase is the most imperative redox partner of multiple P450s involved in primary and secondary metabolite biosynthesis, the expression of heterologous P450 systems is of critical importance in attempts to construct new metabolic pathways in recombinant host cells and in the production of new biocatalysts.

However, heterologous expression of membrane-anchored cytochrome P450 enzymes (CYPs) in microbial hosts for application in biocatalytic processes is challenging. CYP activity is irrevocably linked to a finely balanced system of NADPH cofactor recycling, oxygen supply, correct integration of heme iron into the active site of the CYP and, of course, to perfect interaction with their corresponding cytochrome P450 reductase (CPR) that functions as electron donor.

### SUMMARY OF THE INVENTION

The present invention is based on the finding that cytochrome P450 reductase (CPR) is prone to degradation in a Δice2 knockout strain of yeast. Over-expressing Ice2p increased HPO/CPR-mediated bioconversion of (+)-valencene up to 1.4-fold in resting cell assays.

General applicability of ICE2 overexpression in stabilizing CYP/CPR activities for biocatalytic applications and fermentative production was demonstrated by extending the analysis to alternative CYP/CPR combinations and to both S. cerevisiae and P. pastoris as expression hosts. CPR stabilization was detected throughout.

Biocatalytic application typically refers to use of a CYP/CPR to convert, by enzymatic reaction, one compound to another, whereas fermentative production typically refers to conversion of a C-source to a desired product.

Accordingly, the invention relates to a recombinant host cell which is capable of expressing a cytochrome P450 reductase (CPR) and, optionally, a cytochrome P450 enzyme (CYP) and which is capable of overexpressing a Ice2p comprising the amino acid sequence set out in SEQ ID NO: 2 or a sequence having at least 50% sequence identity thereto.

The invention also relates to:
a method for the production of a compound of interest in a recombinant host cell, which method comprises:
   providing a recombinant host cell according to any one of claims which is capable of expressing a compound of interest;
   cultivating the recombinant host cell under conditions suitable for production of the compound of interest; and, optionally
   recovering the compound of interest; and a method for the production of a compound of interest in a biocatalytic reaction, which method comprises:
   providing a recombinant host cell according to any one of the preceding claims which is capable of producing a CYP and CPR of interest;
   cultivating the recombinant host cell under conditions suitable for production of CYP and CPR;
   converting a suitable substrate to a compound of interest by contacting the substrate with the recombinant host cell or a biocatalyst formulation derived from it; and, optionally
   recovering the compound of interest.

The invention further provides use of Ice2p to stabilize expression of a cytochrome P450 reductase in a recombinant host cell.

### BRIEF DESCRIPTION OF THE DRAWINGS

FIG. 1 sets out (+)-valencene (1) biohydroxylation by CYP/CPR activity recombinantly expressed in S. cerevisiae. trans-nootkatol (2) formed by cytochrome P450 enzymes may be further oxidized to (+)-nootkatone (3) by an unidentified intrinsic activity of baker's yeast (A). Effectors of HPO/CPR activity in S. cerevisiae were screened by resting cells assays with a theoretically re-extractable amount (TRA) of 817 ng of (+)-valencene per μL of ethyl acetate (B). BY4742 (evc) indicates substrate loss within 20 h of assay. BY4742 and single knockout strains disrupted for Δice2, Δyor1 and Δayr1 in the same background were transformed with pYES-HPO-CPR to be assayed for (+)-valencene conversion. Results are given as mean values and standard deviations of resting cells assays performed in technical quadruplicate for two biological replicas. Western blot analyses were performed to reveal HPO/CPR levels after 6 h of galactose induction (C).

FIG. 2 sets out GC-MS chromatograms of reference standards (A) and terpenoids produced by resting cells assay (B); 1, (+)-valencene; 2, cis-nootkatol; 3, trans-nootkatole; 4, (+)-nootkatone. Mass spectra of trans-nootkatol and (+)-nootkatone are shown for reference standards (C, E) and biotransformation products with resting cells (D, F).

FIG. 3 sets out assessment of the effect of modulated ICE2 expression levels on HPO/CPR-mediated conversion of (+)-valencene. Quantitative real-time PCR analysis (A). Control strain W303 and control strain, Δice2 and PGAL1-ICE2 strains co-expressing HPO and CPR from the pYES2 vector. mRNA levels were determined according to the ΔCt method normalizing expression levels to the housekeeping gene CDC73. Same strains were used for resting cells activity assays performed in three biological and four technical replicas each (B).

FIG. 4 sets out in vivo production and bioconversion of (+)-valencene in cells co-expressing ValS, tHMG1, HPO and CPR (2) and strains additionally harboring an deletion of ice2 (3) or overexpressing ICE2 from the PGAL1 promoter (4). Strains were compared to the control strain W303 (1). Conversions were done in triplicates and consistently repeated for more than three independent experiments.

FIG. 5 sets out time-dependent analysis of in vivo production of terpenoids of the reference strain W303 tHMG1 ValS HPO CPR and the strain over-expressing ICE2 (A). Quantification of Western blot signals was done with the Fiji program from samples taken at time points 24 h, 48 h and 72 h. Intermediate band intensities were calculated in percentages from four samples loaded per strain and timepoint (B). At the same time points, expression of Ice2p-His6 was tracked either from the endogenous promoter or from the PGAL1-promoter (1 and 2 OD600 units loaded, respectively) (C). Cytochrome c reductase activity assay was done with the control strain W303 tHMG1 ValS HPO CPR and the one over-expressing ICE2-His6. Background reductase activities of strains W303 MATa and W303 MATα PGal1-ICE2-His6 were subtracted. Measurements were done in quadruplicates with samples taken from two different cultivations (D).

FIG. 6 sets out electron micrographs of S. cerevisiae strains. Subcellular compartments were traced in different colors for better visualization. Nuclear membrane, light grey (N, nucleus); peripheral ER, dark grey. The wild type strain W303 (A), the Δice2 (B) and the strain overexpressing ICE2(C) with and without co-expression of HPO and CPR are shown.

FIG. 7 sets out alternative CYP/CPR systems tested for substrate conversion in S. cerevisiae and P. pastoris strains overexpressing ICE2. S. cerevisiae resting cells assays for conversion of (−)-limonene (PM17/CPR) (A) and bufuralol (CYP2D6/hCPR) (B). Western blot analysis of S. cerevisiae strains expressing alternative pairs of CYPs and reductases were performed (C). As alternative expression host, P. pastoris was tested for both, (−)-limonene (D) and bufuralol (E) conversion. Strains were analyzed for expression of HPO and CPR by Western blot (F).

FIG. 8 sets out results of biphasic (+)-valencene whole-cell hydroxylation assay using P. pastoris strains HCV (expressing ValS, HPO and CPR) and HCV-PpIce2, additionally co-expressing PpIce2 (A). Immunological detection of HPO-flag and CPR-myc proteins in HCV and HCV-PpIce2 strains.

### DESCRIPTION OF THE SEQUENCE LISTING

SEQ ID NO: 1 sets out the mRNA sequence of the ICE2 gene from Saccharomyces cerevisiae 5288c (NM_001179438).

SEQ ID NO: 2 sets out the amino acid sequence of the Ice2p protein from Saccharomyces cerevisiae 5288c (NP_012176).

SEQ ID NOs: 3 to 35 set out the sequences of the primers described in Table 2.

### DETAILED DESCRIPTION OF THE INVENTION

The invention relates to the use of Ice2p to stabilize a cytochrome P450 reductase. Cytochrome P450 enzymes (CYP) together with cytochrome P450 reductases (CPR) constitute important biocatalysts which can be used in vivo, for example in metabolic engineering strategies or which can be used as biocatalysts per se, i.e. contacted with a compound to convert it into a further compound of interest.

However, expression of CYPs and CPRs is not straight-forward. Herein, it is demonstrated that over-expression of the ICE2 gene from S. cerevisiae stabilizes expression of CPRs. This may allow for the generation of better engineered metabolic pathways and/or for the production of better biocatalysts.

Accordingly, the invention relates to a recombinant host cell which is capable of overexpressing or which overexpresses Ice2p comprising the amino acid sequence set out in SEQ ID NO: 2 or an amino acid sequence having at least 50% thereto.

Such a recombinant host cell of the invention will typically also be capable of expressing a CYP and/or a CPR.

Herein, a cytochrome P450 is any member of the cytochrome P450 superfamily of monooxygenases (officially abbreviated as CYP) that catalyze the oxidation of organic substances.

The most common reaction catalyzed by CYPs is a monooxygenase reaction, e.g., insertion of one atom of oxygen into the aliphatic position of an organic substrate (RH) while the other oxygen atom is reduced to water:

RH+02+NADPH+H⁺→ROH+H₂O+NADP⁺

Herein, a cytochrome P450 reductase is any cytochrome P450 reductase (EC 1.6.2.4; also known as NADPH:ferrihemoprotein oxidoreductase, NADPH:hemoprotein oxidoreductase, NADPH:P450 oxidoreductase, P450 reductase, POR, CPR, CYPOR), a membrane-bound enzyme required for electron transfer to cytochrome P450 in the endoplasmic reticulum of the eukaryotic cell from a FAD- and FMN-containing enzyme NADPH:cytochrome P450 reductase (POR; EC 1.6.2.4).

Ice2p is a type III membrane protein with eight predicted transmembrane domains and an essential role in ER distribution, localization and inheritance in budding yeast. The amino acid sequence of the Ice2p protein from S. cerevisiae is set out in SEQ ID NO: 2. The mRNA sequence of the ICE2 gene is set out in SEQ ID NO: 1.

An Ice2p/ICE2 from an origin other than S. cerevisiae may be used, for example P. pastoris or some other suitable source.

The Ice2p which may be overexpressed in a recombinant host cell of the invention may be any protein comprising the amino acid sequence set out in SEQ ID NO: 2 or a protein comprising an amino acid sequence having at least 50% sequence identity with SEQ ID NO: 2. Alternatively, the Ice2p overexpressed in a recombinant host cell of the invention may comprise an amino acid sequence having at least 60%, at least 65%, at least 70%, at least 75%, at least 80%, at least 85%, at least 90%, at least 95%, at least 96%, at least 97%, at least 98% or at least 99% sequence identity to that of SEQ ID NO: 2 (or be substantially identical to the amino acid sequence of SEQ ID NO: 2).

Any Ice2p protein or variant Ice2P protein overexpressed in a recombinant host cell of the invention will retain Ice2p activity. For the purposes of this invention, a Ice2p protein or variant Ice2p protein overexpressed in a recombinant host of the invention is one which retains at least one activity or property of an Ice2p protein, for example the Ice2p protein having the sequence set out in SEQ ID NO: 2. Thus, a Ice2p protein or variant Ice2p protein overexpressed in a recombinant host of the invention will typically be a type III membrane protein, for example a protein with eight predicted transmembrane proteins. Such a protein may be an ER-located integral membrane protein.

A host cell of the invention is a recombinant host cell. “Recombinant” in this sense means that the host cell is a non-naturally occurring host cell, for example modified by introduction of one or more nucleic acids using recombinant techniques. A nucleic acid used to modify a host cell to arrive at a recombinant host cell of the invention may be a naturally-occurring nucleic acid or a non-naturally occurring nucleic acid. Thus, when used in reference to a host cell of the invention, “recombinant” indicates that the cell has been modified by the introduction of a heterologous nucleic acid or protein or the alteration of a native nucleic acid or protein, or that the cell is derived from a cell so modified. Thus, for example, a recombinant cell may express an Ice2p protein not found within the native (non-recombinant) form of the cell or may be modified so as to express a native gene encoding an Ice2 protein to a greater degree than takes place within the native “non-recombinant” form of the cell. The term “recombinant” is synonymous with “genetically modified”.

A host cell of the invention is thus typically recombinant at least in relation to the overexpression of a Ice2p protein. For example, a recombinant host cell of the invention may comprise a heterologous nucleic acid encoding a Ice2p protein—the heterologous nucleic acid may encode a Ice2p protein which is non-native or native to the non-recombinant form of the cell. That is to say, overexpression of a Ice2p protein may be achieved by introduction of one or more copies of a non-native Ice2p gene or one or more additional copies of a native Ice2p gene or a combination thereof. Alternatively, or in addition, the recombinant host cell of the invention may comprise a modification within a native gene such that the gene is expressed to a greater degree than in a non-recombinant form of the cell and/or the modified such that the resulting Ice2p protein has a higher activity than in a non-recombinant form of the cell.

In a host cell of the invention, a Ice2p protein is overexpressed. Herein, “overexpressed”, “overexpression” or the like implies that the recombinant host cell expresses more of the Ice2p protein than a corresponding cell which does not overexpress a Ice2p protein or, alternatively, that the Ice2p protein is expressed in a cell which would not typically express that protein. Alternatively, overexpression may be achieved by expressing a variant Ice2p protein having a higher specific activity.

Overexpression of a Ice2p protein may equally be referred to as overexpression of an ICE2 gene.

In a recombinant host cell according to the invention, the CYP may be premnaspirodiene oxygenase CYP71D55 from Hyoscymaus muticus (HPO), optionally in which the mutations V482I and A484I have been made, (−)-limonene-3-hydroxylase from Mentha piperita (PM17) or human cytochrome P450 2D6 (CYP2D6).

In a recombinant host cell of the invention, the CPR may be a cytochrome P450 reductase from A. thaliana or a human reductase. The CPY may be a cytochrome P450 reductase having the amino acid sequence set out in any one of SEQ ID NOs: 54, 56, 58 or 78 in WO2013/110673.

A recombinant host cell of the invention is typically capable of production of a compound of interest. The compound of interest may be encoded, wholly or in part, by the one or more recombinant polynucleotides (i.e. polynucleotides introduced into the recombinant host cell by recombinant means). Such polynucleotides may be part of a pathway leading to production of the compound of interest. The compound of interest may, however, be a CYP and/or CPR. Also, the compound of interest may be biomass itself, i.e. the host cell.

Thus, the compound of interest can be any biological compound. The biological compound may be biomass or a biopolymer or metabolite. The biological compound may be encoded by a single polynucleotide or a series of polynucleotides composing a biosynthetic or metabolic pathway or may be the direct result of the product of a single polynucleotide or products of a series of polynucleotides. The biological compound may be native to the host cell or heterologous.

The term “heterologous biological compound” is defined herein as a biological compound which is not native to the cell; or a native biological compound in which structural modifications have been made to alter the native biological compound.

Typically, the compound of interest will be one in which the activity of one or more CYP and/or CPR is involved.

In a recombinant host cell of the invention, the compound of interest may be a sterol, for example 7-dehydrocholesterol or 25-hydroxy 7-dehydrocholesterol, a vitamin, for example vitamin D3, trans-nootkatol, nootkatone or a steviol glycoside, for example steviolmonoside, steviolbioside, stevioside, rebaudioside A, rebaudioside B, rebaudioside C, rebaudioside D, rebaudioside E, rebaudioside F, rebaudioside M, rubusoside or dulcoside A.

Accordingly, the invention relates to use of Ice2p in the production of 7-dehydrocholesterol or 25-hydroxy 7-dehydrocholesterol, a vitamin, for example vitamin D3, trans-nootkatol, nootkatone or a steviol glycoside, for example steviolmonoside, steviolbioside, stevioside, rebaudioside A, rebaudioside B, rebaudioside C, rebaudioside D, rebaudioside E, rebaudioside F, rebaudioside M, rubusoside or dulcoside A. Such production may be fermentative (i.e. production by a recombinant host cell of the invention) or biocatalytic (i.e. by contacting a suitable substrate with a recombinant host cell of the invention or with a biocatalyst derived from such a recombinant host cell).

The invention also relates to use of Ice2p to stabilize CPR in the conversion of valencene to trans-nootkatone and/or in the conversion of trans-nootkatone to nootkatone.

Thus, a recombinant host cell of the invention may be one which has been modified so as to produce one of the above mentioned products of interest.

Ice2p may be overexpressed in a recombinant microorganism as described in WO2013/110673 (in the context of steviol glycoside production) or in a yeast as described in WO2011/067144 (in the context of sterol production).

In the context of the present invention a “host cell” according to the invention or a parent of said host cell may be any type of host cell. A host cell according to any one of the preceding claims wherein the host cell is a eukaryotic or a prokaryotic cell.

The host cell may be a prokaryotic cell. Preferably, the prokaryotic host cell is bacterial cell. The term “bacterial cell” includes both Gram-negative and Gram-positive microorganisms. Suitable bacteria may be selected from e.g. Escherichia, Anabaena, Caulobactert, Gluconobacter, Rhodobacter, Pseudomonas, Paracoccus, Bacillus, Brevibacterium, Corynebacterium, Rhizobium (Sinorhizobium), Flavobacterium, Klebsiella, Enterobacter, Lactobacillus, Lactococcus, Methylobacterium, Staphylococcus or Streptomyces. Preferably, the bacterial cell is selected from the group consisting of B. subtilis, B. amyloliquefaciens, B. licheniformis, B. puntis, B. megaterium, B. halodurans, B. pumilus, G. oxydans, Caulobactert crescentus CB 15, Methylobacterium extorquens, Rhodobacter sphaeroides, Pseudomonas zeaxanthinifaciens, Pseudomonas fluorescens, Paracoccus denitrificans, E. coli, C. glutamicum, Staphylococcus camosus, Streptomyces lividans, Sinorhizobium melioti and Rhizobium radiobacter.

In the invention, the host cell may be a prokaryotic cell, preferably a bacterial cell, more preferably a bacterial cell belonging to the genus Bacillus, Escherichia (such as Escherichia coli), Pseudomonas, Lactobacillus.

A suitable bacterial host cell may additionally contain modifications, e.g. the bacterial host cell may be deficient in genes which are detrimental to the production, recovery and/or application of the compound of interest, e.g. a compound of interest being a polypeptide, e.g. an enzyme. In a preferred aspect the bacterial host cell is a protease deficient host cell, more preferably it is a Bacillus host cell deficient in the gene aprE coding for extracellular alkaline protease and deficient in the gene nprE coding for extracellular neutral metalloprotease. In another preferred aspect the Bacillus host cell is further deficient in one or more proteases coded by the genes selected from the group consisting of: nprB, vpr, epr, wprA, mpr, bpr. In another preferred aspect the bacterial host cell does not produce spores and or is deficient in a sporulation related gene such as e.g. spo0A, spoIISA, sigE, sigF, spoIISB, spoIIE, sigG, spoIVCB, spoIIIC, spoIIGA, spoIIAA, spoIVFB, spoIIR, spoIIIJ. In yet another preferred aspect the Bacillus host cell is deficient in the gene amyE coding for α-amylase. In yet another preferred aspect the Bacillus host cell, more preferably a Bacillus subtilis host cell, is deficient in aprE, nprE, amyE and does not produce spores. In a more preferred embodiment the Bacillus host cell is BS154, CBS136327 or a derivative thereof.

A host cell according to the invention may be a eukaryotic host cell. Preferably, the eukaryotic cell is a mammalian, insect, plant, fungal, or algal cell such as a Schizochitrium. Preferred mammalian cells include e.g. Chinese hamster ovary (CHO) cells, COS cells, 293 cells, PerC6 cells, and hybridomas. Preferred insect cells include e.g. Sf9 and Sf21 cells and derivatives thereof. More preferably, the eukaryotic cell is a fungal cell, e.g. a yeast cell or a filamentous fungal cell.

A preferred yeast host cell may be from the genus Candida, Hansenula, Issatchenkia, Kluyveromyces, Pichia, Saccharomyces, Schizosaccharomyces, Yarrowia or Zygosaccharomyces. More preferably a yeast host cell is selected from the group consisting of Kluyveromyces lactis, Kluyveromyces lactis NRRL Y-1140, Kluyveromyces marxianus, Kluyveromyces thermotolerans, Candida krusei, Candida sonorensis, Candida glabrata, Saccharomyces cerevisiae, Saccharomyces cerevisiae CEN.PK113-7D, Schizosaccharomyces pombe, Hansenula polymorpha, Issatchenkia orientalis, Yarrowia lipolytica, Yarrowia lipolytica CLIB122, Yarrowia lipolytica ML324 (deposited as ATCC18943), Pichia stipidis and Pichia pastoris.

A host cell may be a filamentous fungal cell. Filamentous fungi as defined herein include all filamentous forms of the subdivision Eumycota and Oomycota (as defined by Hawksworth et al., In, Ainsworth and Bisby's Dictionary of The Fungi, 8th edition, 1995, CAB International, University Press, Cambridge, UK). The filamentous fungal host cell may be a cell of any filamentous form of the taxon Trichocomaceae (as defined by Houbraken and Samson in Studies in Mycology 70: 1-51. 2011). In another preferred embodiment, the filamentous fungal host cell may be a cell of any filamentous form of any of the three families Aspergillaceae, Thermoascaceae and Trichocomaceae, which are accommodated in the taxon Trichocomaceae. The filamentous fungi are characterized by a mycelial wall composed of chitin, cellulose, glucan, chitosan, mannan, and other complex polysaccharides. Vegetative growth is by hyphal elongation and carbon catabolism is obligately aerobic. Filamentous fungal strains include, but are not limited to, strains of Acremonium, Agaricus, Aspergillus, Aureobasidium, Chrysosporium, Coprinus, Cryptococcus, Filibasidium, Fusarium, Humicola, Magnaporthe, Mortierella, Mucor, Myceliophthora, Neocaffimastix, Neurospora, Paecilomyces, Peniciffium, Piromyces, Panerochaete, Pleurotus, Schizophyllum, Talaromyces, Rasamsonia, Thermoascus, Thielavia, Tolypocladium, and Trichoderma.

Preferred filamentous fungal cells belong to a species of an Acremonium, Aspergillus, Chrysosporium, Myceliophthora, Penicillium, Talaromyces, Rasamsonia, Thielavia, Fusarium or Trichoderma genus, and most preferably a species of Aspergillus niger, Acremonium alabamense, Aspergillus awamori, Aspergillus foetidus, Aspergillus sojae, Aspergillus fumigatus, Talaromyces emersonii, Rasamsonia emersonii, Aspergillus oryzae, Chrysosporium lucknowense, Fusarium oxysporum, Myceliophthora thermophila, Trichoderma reesei, Thielavia terrestris or Penicillium chrysogenum. A more preferred filamentous fungal host cell belongs to the genus Aspergillus, more preferably the host cell belongs to the species Aspergillus niger. When the host cell according to the invention is an Aspergillus niger host cell, the host cell preferably is CBS 513.88, CB5124.903 or a derivative thereof.

Several microbial strains are readily accessible to the public in a number of culture collections, such as the American Type Culture Collection (ATCC), Deutsche Sammlung von Mikroorganismen und Zellkulturen GmbH (DSM), Centraalbureau Voor Schimmelcultures (CBS), Agricultural Research Service Patent Culture Collection, Northern Regional Research Center (NRRL), and All-Russian Collection of Microorganisms of Russian Academy of Sciences, (abbreviation in Russian—VKM, abbreviation in English—RCM), Moscow, Russia. Useful strains in the context of the present invention may be Aspergillus niger CBS 513.88, CB5124.903, Aspergillus oryzae ATCC 20423, IFO 4177, ATCC 1011, CB5205.89, ATCC 9576, ATCC14488-14491, ATCC 11601, ATCC12892, P. chrysogenum CBS 455.95, P. chrysogenum Wisconsin54-1255(ATCC28089), Penicillium citrinum ATCC 38065, Penicillium chrysogenum P2, Thielavia terrestris NRRL8126, Talaromyces emersonii CBS 124.902, Acremonium chrysogenum ATCC 36225 or ATCC 48272, Trichoderma reesei ATCC 26921 or ATCC 56765 or ATCC 26921, Aspergillus sojae ATCC11906, Myceliophthora thermophila C1, Garg 27K, VKM-F 3500 D, Chrysosporium lucknowense C1, Garg 27K, VKM-F 3500 D, ATCC44006 and derivatives thereof.

The invention also relates to a method for the production of a compound of interest in a recombinant host cell, which method comprises:

providing a recombinant host cell according to any one of claims which is capable of expressing a compound of interest;

cultivating the recombinant host cell under conditions suitable for production of the compound of interest; and, optionally

recovering the compound of interest.

The invention further relates to a method for the production of a compound of interest in a biocatalytic reaction, which method comprises:

providing a recombinant host cell according to any one of the preceding claims which is capable of producing a CYP and CPR of interest;

cultivating the recombinant host cell under conditions suitable for production of CYP and CPR;

converting a suitable substrate to a compound of interest by contacting the substrate with the recombinant host cell or a biocatalyst formulation derived from it; and, optionally

recovering the compound of interest.

Either of these methods may be applied to the conversion of valencene to trans-nootkatone and/or in the conversion of trans-nootkatone to nootkatone. The reaction scheme for this is shown below:

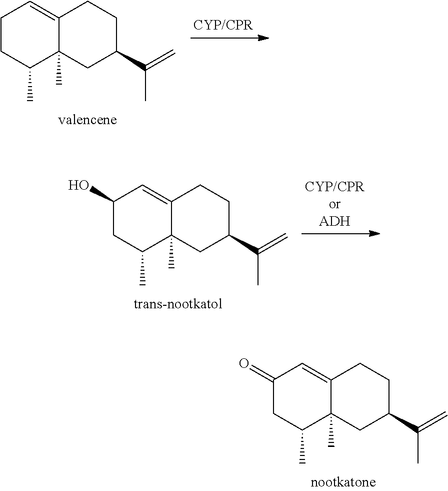

That is to say, the invention also relates to use of Ice2p to stabilize CPR in the conversion of valencene to trans-nootkatone and/or in the conversion of trans-nootkatone to nootkatone. A recombinant host cell of the invention may be used in a process for conversion of valencene to trans-nootkatone and/or in the conversion of trans-nootkatone to nootkatone, either by use of the recombinant cell itself or by use of a biocatalyst derived from the recombinant cell.

Also provided by the invention is use of Ice2p to stabilize expression of a cytochrome P450 reductase in a recombinant host cell. In particular, overexpression of Ice2p may be used to stabilize expression of a CPR in a host cell.

The method of the invention for the preparation of a compound of interest comprises cultivating, i.e. fermenting, a recombinant cell as described herein, in the presence of a suitable fermentation medium under appropriate conditions. Suitable fermentation media are known to the person skilled in the art. Fermentation of the recombinant cell may be carried out under conditions which lead to the production of the compound of interest. The method of the invention may be carried out in the presence or absence of oxygen. That is, the method is carried out under anaerobic conditions.

For the purposes of this invention, an anaerobic fermentation process may be herein defined as a fermentation process run in the absence of oxygen or in which substantially no oxygen is consumed, preferably less than 5, 2.5 or 1 mmol/L/h, and wherein organic molecules serve as both electron donor and electron acceptors. The fermentation process according to the present invention may also first be run under aerobic conditions and subsequently under anaerobic conditions. Anaerobic conditions are typically used in the production phase (production of the compound of interest).

The fermentation process of the invention may also be run under oxygen-limited, or micro-aerobic, conditions which are, for the purposes of this invention, considered to be anaerobic processes. Alternatively, the fermentation process may first be run under aerobic conditions and subsequently under oxygen-limited conditions. An oxygen-limited fermentation process is a process in which the oxygen consumption is limited by the oxygen transfer from the gas to the liquid. The degree of oxygen limitation is determined by the amount and composition of the ingoing gasflow as well as the actual mixing/mass transfer properties of the fermentation equipment used.

The process for the production of a compound of interest according to the present invention may be carried out at any suitable pH between 1 and 9. Preferably, the pH in the fermentation broth is between 2 and 7.

A suitable temperature at which the process according to the present invention may be carried out is between 5 and 60° C., preferably between 10 and 50° C., more preferably between 15 and 35° C., more preferably between 18° C. and 30° C. The person skilled in the art knows which optimal temperatures are suitable for fermenting a specific recombinant cell.

In a method of the invention for the production of a compound of interest, the compound of interest may be secreted into the fermentation broth and/or present in the recombinant cell used in the invention. Thus, the invention also provides a fermentation broth comprising the compound of interest obtainable by a method according to the invention. The broth may comprise: the compound of interest which has been secreted from a recombinant cell: the compound of interest comprised within a recombinant cell of the invention: the compound of interest which has been released from a recombinant cell following treatment of the cell to cause disruption of the cell and release of the compound of interest; or a mixture of any thereof.

Preferably, in the method of the invention: the compound of interest is recovered from the fermentation broth by a suitable method known in the art, for instance by extraction or crystallisation. A recombinant cell of the invention may need to be disrupted to allow for release of the compound of interest.

The term “stabilize” in the context of the present invention means that the expression of the cytochrome P450 reductase is, for example increased and/or prolonged in comparison with a form of the host cell which does not overexpressa Oce2p protein. This may be apparent in view of increased production of a compound of interest.

The terms “sequence identity” or “sequence homology” are used interchangeably herein. For the purpose of this invention, it is defined here that in order to determine the percentage of sequence identity or sequence homology of two amino acid sequences or of two nucleic acid sequences, the sequences are aligned for optimal comparison purposes. In order to optimize the alignment between the two sequences, gaps may be introduced in any of the two sequences that are compared. Such alignment can be carried out over the full length of the sequences being compared. Alternatively, the alignment may be carried out over a shorter length, for example over about 20, about 50, about 100 or more nucleic acids/based or amino acids. The sequence identity is the percentage of identical matches between the two sequences over the reported aligned region.

A comparison of sequences and determination of percentage of sequence identity between two sequences can be accomplished using a mathematical algorithm. The skilled person will be aware of the fact that several different computer programs are available to align two sequences and determine the identity between two sequences (Kruskal, J. B. (1983) An overview of sequence comparison In D. Sankoff and J. B. Kruskal, (ed.), Time warps, string edits and macromolecules: the theory and practice of sequence comparison, pp. 1-44 Addison Wesley). The percent sequence identity between two amino acid sequences or between two nucleotide sequences may be determined using the Needleman and Wunsch algorithm for the alignment of two sequences. (Needleman, S. B. and Wunsch, C. D. (1970) J. Mol. Biol. 48, 443-453). Both amino acid sequences and nucleotide sequences can be aligned by the algorithm. The Needleman-Wunsch algorithm has been implemented in the computer program NEEDLE. For the purpose of this invention the NEEDLE program from the EMBOSS package was used (version 2.8.0 or higher, EMBOSS: The European Molecular Biology Open Software Suite (2000) Rice, P. Longden, I. and Bleasby, A. Trends in Genetics 16, (6) pp 276-277, http://emboss.bioinformatics.nl/). For protein sequences EBLOSUM62 is used for the substitution matrix. For nucleotide sequence, EDNAFULL is used. The optional parameters used are a gap-open penalty of 10 and a gap extension penalty of 0.5. The skilled person will appreciate that all these different parameters will yield slightly different results but that the overall percentage identity of two sequences is not significantly altered when using different algorithms.

After alignment by the program NEEDLE as described above the percentage of sequence identity between a query sequence and a sequence of the invention is calculated as follows: Number of corresponding positions in the alignment showing an identical amino acid or identical nucleotide in both sequences divided by the total length of the alignment after subtraction of the total number of gaps in the alignment. The identity defined as herein can be obtained from NEEDLE by using the NOBRIEF option and is labeled in the output of the program as “longest-identity”.

The protein sequences and nucleic acid sequences referred to herein can further be used as a “query sequence” to perform a search against public databases to, for example, identify other family members or related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to nucleic acid molecules of the invention. BLAST protein searches can be performed with the XBLAST program, score=50, wordlength=3 to obtain amino acid sequences homologous to protein molecules of the invention. To obtain gapped alignments for comparison purposes, Gapped BLAST can be utilized as described in Altschul et al., (1997) Nucleic Acids Res. 25(17): 3389-3402. When utilizing BLAST and Gapped BLAST programs, the default parameters of the respective programs (e.g., XBLAST and NBLAST) can be used. See the homepage of the National Center for Biotechnology Information at http://www.ncbi.nlm.nih.gov/.

Standard genetic techniques, such as overexpression of enzymes in host cells, genetic modification of host cells, or hybridisation techniques, are known methods in the art, such as described in Sambrook and Russel (2001) “Molecular Cloning: A Laboratory Manual (3^{rd} edition), Cold Spring Harbor Laboratory, Cold Spring Harbor Laboratory Press, or F. Ausubel et al, eds., “Current protocols in molecular biology”, Green Publishing and Wiley Interscience, New York (1987). Methods for transformation, genetic modification etc. of fungal host cells are known from e.g. EP-A-0 635 574, 98/46772, WO 99/60102 and WO 00/37671, WO90/14423, EP-A-0481008, EP-A-0635 574 and U.S. Pat. No. 6,265,186. Accordingly, preparation of a recombinant cell suitable for use in the invention is well known to those skilled in the art.

### Embodiments of the Invention

1. A recombinant host cell which is capable of expressing a cytochrome P450 enzyme (CYP) and a cytochrome P450 reductase (CPR) and which is capable of overexpressing a Ice2p comprising the amino acid sequence set out in SEQ ID NO: 2 or a sequence having at least 50% sequence identity thereto.
2. A recombinant host cell according to embodiment 1, wherein the CYP is premnaspirodiene oxygenase CYP71D55 from Hyoscymaus muticus (HPO), optionally in which the mutations V482I and A484I have been made, (−)-limonene-3-hydroxylase from Mentha piperita (PM17) or human cytochrome P450 2D6 (CYP2D6).
3. A recombinant host cell according to embodiment 1 or 2, wherein the CRP is a cytochrome P450 reductase from A. thaliana or a human reductase.
4. A recombinant host cell according to any one of the preceding embodiments which is capable of production of a compound of interest.
5. A recombinant host cell according to embodiment 4, wherein the compound of interest is a sterol, a steviol glycoside, trans-nootkatol or nootkatone.
6. A recombinant host cell according to any one of the preceding embodiments wherein the host cell is a eukaryotic or a prokaryotic cell.
7. A recombinant host cell according to embodiment 6 which is a eukaryotic cell, preferably a fungal cell, more preferably a yeast cell selected from the group consisting of Candida, Hansenula, Kluyveromyces, Pichia, Saccharomyces, Schizosaccharomyces, or Yarrowia strains, or a filamentous fungal cell selected from the group consisting of filamentous fungal cells belong to a species of Acremonium, Aspergillus, Chrysosporium, Myceliophthora, Penicillium, Talaromyces, Rasamsonia, Thielavia, Fusarium or Trichoderma.
8. A recombinant host cell according to embodiment 6 which is a prokaryotic cell, preferably a bacterial cell, more preferably a bacterial cell belonging to the genus Bacillus, Escherichia, Pseudomonas, Lactobacillus.
9. A method for the production of a compound of interest in a recombinant host cell, which method comprises:
   providing a recombinant host cell according to any one of the preceding embodiments which is capable of expressing a compound of interest;
   cultivating the recombinant host cell under conditions suitable for production of the compound of interest; and, optionally
   recovering the compound of interest.
10. A method for the production of a compound of interest in a biocatalytic reaction, which method comprises:
   providing a recombinant host cell according to any one of the preceding embodiments which is capable of producing a CYP and CPR of interest;
   cultivating the recombinant host cell under conditions suitable for production of CYP and CPR;
   converting a suitable substrate to a compound of interest by contacting the substrate with the recombinant host cell or a biocatalyst formulation derived from it; and, optionally
   recovering the compound of interest.
11. A method according to embodiment 9 or 10 for the conversion of valencene to trans-nootkatone and/or in the conversion of trans-nootkatone to nootkatone
12. Use of Ice2p to stabilize expression of a cytochrome P450 reductase in a recombinant host cell.

Throughout the present specification and the accompanying claims, the words “comprise”, “include” and “having” and variations such as “comprises”, “comprising”, “includes” and “including” are to be interpreted inclusively. That is, these words are intended to convey the possible inclusion of other elements or integers not specifically recited, where the context allows.

The articles “a” and an are used herein to refer to one or to more than one (i.e. to one or at least one) of the grammatical object of the article. By way of example, “an element” may mean one element or more than one element.

A reference herein to a patent document or other matter which is given as prior art is not to be taken as an admission that that document or matter was known or that the information it contains was part of the common general knowledge as at the priority date of any of the claims.

The disclosure of each reference set forth herein is incorporated herein by reference in its entirety.

The present invention is further illustrated by the following Examples:

### EXAMPLES

### Materials and Methods

### Chemicals

Unless stated otherwise, standard laboratory reagents were obtained from Sigma-Aldrich® (Steinheim, Germany) or Carl Roth GmbH & Co. KG (Karlsruhe, Germany) with the highest purity available. The pYES2 expression vector was purchased from Invitrogen (Carlsbad, USA). The pESC-URA expression vector was obtained from Agilent Technologies (Santa Clara, USA). Restriction enzymes were acquired from Thermo Scientific (St. Leon-Rot, Germany). Difco™ yeast nitrogen base w/o amino acids (YNB), Bacto™ tryptone and Bacto™ yeast extract were obtained from Becton Dickinson and Company (Schwechat, Austria). Geneticin sulfate (G418), Hygromycin B and Kanamycin monosulfate were ordered from Formedium™ (Norfolk, United Kingdom). Zeocin™ was purchased from InvivoGen (Eubio) (Vienna, Austria). Sterile water was purchased from Fresenius Kabi (Graz, Austria). Terpenoid standards were supplied by DSM Innovative Synthesis B.V. (Geleen, The Netherlands).

### 1.1 Microorganisms, Plasmids and Media

For all cloning steps and plasmid replication, E. coli Top 10 F′ (F′[lacl^{q} Tn10(tet^{R})] mcrA Δ(mrr-hsdRMS-mcrBC) φ80lacZΔM15 ΔlacX74 deoR nupG recA1 araD139 Δ(ara-leu)7697 galU galK rpsL(Str^{R}) endA1λ⁻) from life technologies (Vienna, Austria) was used. S. cerevisiae strains were constructed either in the BY4742 (MATα, his3-1, leu2-0, lys2-0, ura3-0) or in the W303 (MATa and MATα, ade2-1, trp1-1, can1-100, leu2-3, 112, his3-11, 15, ura3-1, GALs⁺) background. BY4742 and single gene knockout strains thereof were obtained from the EUROSCARF collection (http://web.uni-frankfurt.de/fb15/mikro/euroscarf/).

S. cerevisiae strains were cultivated in synthetic defined media (6.7 g yeast nitrogen base w/o amino acids; 1 g drop-out powder consisting of equal amounts of adenine, lysine, tyrosine, histidine, leucine and tryptophane; 2% glucose). Synthetic defined induction media containing 2% galactose and 0.7% raffinose instead of 2% glucose was used for induction.

P. pastoris cultures were either grown in YPD (1% yeast extract, 2% peptone and 2% glucose) or buffered complex glycerol medium, BMGY (1% yeast extract, 2% peptone, 100 mM potassium phosphate, pH 6.0, 1.34% YNB, 4×10⁻⁵% biotin, 1% glycerol). BMMY (1% yeast extract, 2% peptone, 100 mM potassium phosphate, pH 6.0, 1.34% YNB, 4×10⁻⁵% biotin, 1% methanol) was used as induction medium.

### Cloning of Expression Vectors and Yeast Strain Generation

A list of strains generated during this study is given in Table 1. Standard molecular cloning technologies were used in strain generation (Ausubel et al., 2003). For gene amplification, Phusion® High Fidelity DNA polymerase (Thermo Fisher Scientific Inc., St. Leon-Rot, Germany) was utilized in accordance with the recommended PCR protocol. Codon optimized gene variants of HPO (H. muticus premnaspirodiene oxygenase), CPR (A. thaliana cytochrome P450 reductase), and ValS (C. nootkatensis terpene synthase), were designed manually and purchased from GeneArt® (Wriessnegger et al. manuscript in revision).

Saccharomyces cerevisiae

For generation of the pYES2 based co-expression construct, the CPR gene was amplified using primers Fw_CPR_HindIII and Rv_CPR-myc_BamHI (Table 2) for HindIII/BamHI cloning into pYES2. The resulting plasmid was modified by introducing the restriction sites for BgIII and AscI using primers pYES2_AscI_fw and pYES2_BgIII_rev, and was re-circularized by ligation with the HPO-Flag expression cassette. The latter was constructed by HindIII/BamHI cloning the HPO-Flag fragment amplified with Fw_HPOSc_HindIII and Rv_HPO-Flag_Sc into pYES2 and, subsequently, by amplifying the whole expression cassette using primers BgIII_Gal1_fw and AscI_CYC1_rev. Knockout of ICE2 was achieved through integration of a kanMX cassette amplified with F1(ICE2) and R1(ICE2) primers from pFA6a-KanMX (Longtine et al., 1998). For expression of chromosomally His-tagged Ice2p, ICE2 (GenBank number: NM_001179438.1) was amplified from genomic DNA of S. cerevisiae W303 using primers Fw(ICE2_XhoI) and Rv(ICE2-6His_XbaI) and was XhoI/XbaI cloned into pYES2. The ICE2 gene placed under the control of the P_{Gal1} promoter and CYC1 termination sequence was amplified using BgIII_Gar1_fw and AscI_CYC1_rev primers and was inserted into pFA6a-TRP1 (Longtine et al., 1998) via BgIII/AscI restriction sites. This construct served as template to generate cassettes for ICE2-His₆ expression from the endogenous promoter using primers F1(ICE2start) and R1 (ICE2), or for over-expression from the P_{Gal1} promoter using primers F1(ICE2) and R1(ICE2). Both expression cassettes were integrated into the genomic ICE2 locus. The codon-optimized (+)-valencene synthase gene was subcloned from the Geneart® delivery vector into pYES2 via EcoRI and BamHI restriction sites. The ValS integration cassette was amplified with BgIII_Gal1_fw and AscI_CYC1_rev primers and BgIII/AscI cloned into the vector pFA6a-HIS3kanMX6 (Longtine et al., 1998). Then, the ValS expression cassette was amplified using primers F1(trp1) and R1(trp1), and was transformed into yeast. For over-expression of truncated HMG1 from the P_{PGK1} promoter, the PGK1 promoter region was amplified from genomic DNA of S. cerevisiae W303 using Fw(P_{PGK1}_{_}XmaI) and Rv(P_{PGK1}_{_}BamHI) primers to be inserted into pFL36-LEU2 (Bonneaud et al., 1991) via XmaI and BamHI restriction sites. Truncated HMG1 (GenBank number: NM_001182434.1) devoid of its sterol sensing domain was amplified from genomic DNA of S. cerevisiae with primers Fw(tHMG1_PstI) and Rv(tHMG1_HindIII) and was cloned into pFL36-LEU2-P_{PGK1} via PstI and HindIII sites. Primers Fw(ex_leu2_tHMG1) and Rv(ex_1 eu2_tHMG1) were applied to amplify the tHMG1 expression cassette including the P_{PGK1} promoter and LEU2 selection marker for integration into the W303 leu2 locus. Transformations of plasmids as well as DNA cassettes was done following the lithium acetate method (Gietz and Schiestl, 1995). Transformants were selected either on synthetic defined plates lacking histidine, uracil or leucine or on YPD plates containing 300 mg L⁻¹ geneticin (Botstein, D., 1982). Correct integration of cassettes into the specific loci was routinely confirmed by colony PCR (Kwiatkowski et al., 1990). For generating triple knockin/knockout mutants, strain W303 MATa P_{PGK1}-tHMG1 P_{GAL1}-ValS was mated with single mutants W303 MATα Δice2::KanMX and W303 MATα P_{GAL1}-ICE2-6His to be sporulated and dissected for single spores harboring all three genes (Amberg et al., 2006).

Codon optimized gene variants of the following genes were designed manually by applying the Pichia pastoris codon usage: limonene-3-hydroxylase (PM17 isoform, CYP71D13, GenBank number of native gene: AF124816), human cytochrome P450 2D6 (GenBank number of native gene: NM_000106) and human cytochrome P450 reductase (GenBank number of native gene: NM_000941) (Suppl. Table 1).

Optimized hCPR was cloned into pESC-URA with EcoRI and NotI by cutting the synthetic gene directly out of the delivered vector. Afterwards, CYP2D6 was cloned into pESC-URA-hCPR with HindIII and BamHI. To construct the co-expression vector for PM17 and CPR, CPR was amplified with Fw(CPR_NotI) and Rv(CPRmyc_BgIII) and cloned into pESC-URA. PM17 was cut out with BamHI and HindIII from the synthetic vector to be ligated with pESC-URA-CPR.

Pichia pastoris

The P. pastoris strain CBS7435 his4 (Naatsaari et al., 2012) was used as host strain for the construction of strains PpPM17/CPR and Pp2D6/hCPR, respectively. Generation of strain PpHCV was recently described in detail by Wriessnegger et al. (manuscript in revision).

Codon optimized PM17, 2D6, CPR and hCPR genes with desired EcoRI/NotI restriction sites for cloning, and C-terminal FLAG- and myc-tags on the CYP450s and CPRs, respectively, were purchased from GeneArt® (Supplemental Table 51). For creation of the P. pastoris PM17/AtCPR co-expression vector the AtCPR and the PM17 genes were subcloned into the EcoRI and NotI digested expression vector pPpB1 containing a synthetic variant of the AOX1 promoter and a Zeocin™ resistance marker cassette for selection. The generated pPpB1[CPR] vector was cut with BgIII and BamHI to obtain the AtCPR gene flanked by AOX1 promoter and terminator regions. The purified fragment was ligated into the BamHI digested vector pPpB1[PM17] to obtain the pPpB1[PM17/AtCPR] co-expression vector. The same strategy was applied for the generation of the pPpB1[2D6/hCPR] co-expression vector. Expression vectors were checked by sequencing the expression cassette and were linearized with BgIII for integration into the genome of P. pastoris.

PpICE2 (PAS_chr2-2_0195) was identified by blasting the ScICE2 protein sequence against the P. pastoris G5115 genome data base. PpICE2 was amplified from genomic DNA of P. pastoris CB57435 using primer pairs FwPpICE2 and RvPpICE2 containing restriction sites EcoRI and NotI, respectively, for cloning into the pPpKan expression vector harboring the AOX1 promoter and the kanamycin/geneticin selection cassette (Table 2). After linearization of the expression vector pPpKan[PpICE2] with BgIII, for transformation into the genome of CYP/CPR co-expressing P. pastoris strains PpHCV, PpPM17/AtCPR and Pp2D6/hCPR, respectively. Routinely, competent P. pastoris cells were transformed with ˜2 μg of linearized plasmids according to the protocol of Lin-Cereghino. After transformation, aliquots were plated on YPD plates containing 100 mg/L Zeocin™ or 400 mg/L geneticin.

TABLE 1
Strains used in this study

| | Genotypes of strains (harboring | |
|---|---|---|
| Strain ID | plasmids) | Source |
| BY4742 pYES2 (empty vector control, | MATα, his3Δ1, leu2Δ0, lys2Δ0, | This study |
| evc) | ura3Δ0 (pYES2) | |
| BY4742 pYES2-HPO-CPR | BY4742 (pYES2 PGAL1-HPO-Flag | This study |
| | PGAL1-CPR-myc) | |
| BY4742 Δice2 pYES2-HPO-CPR | BY4742 ice2::KanMX (pYES2 | This study |
| | PGAL1-HPO-Flag PGAL1-CPR-myc) | |
| BY4742 Δayr1 pYES2-HPO-CPR | BY4742 ayr1::KanMX (pYES2 | This study |
| | PGAL1-HPO-Flag PGAL1-CPR-myc) | |
| BY4742 Δyor1 pYES2-HPO-CPR | BY4742 yor1::KanMX (pYES2 | This study |
| | PGAL1-HPO-Flag PGAL1-CPR-myc) | |
| W303 pYES2 (evc) | MATa or MATα, ade2-1, trp1-1, | This study |
| | can1-100, leu2-3, 112, his3-11, 15, | |
| | ura3-1, GALs+ (pYES2) | |
| W303 Δice2 pYES2-HPO-CPR | W303 MATα Δice2::KanMX (pYES2 | This study |
| | PGAL1-HPO-Flag PGAL1-CPR-myc) | |
| W303 Δice2 (evc) | W303 MATα ice2::KanMX (pYES2) | This study |
| W303 PGAL1-ICE2 pYES2-HPO-CPR | W303 MATα PGAL1-ICE2-His6 | This study |
| | (pYES2 PGAL1-HPO-Flag PGAL1- | |
| | CPR-myc) | |
| W303 PGAL1-ICE2 (evc) | W303 MATα PGAL1-ICE2-His6 | This study |
| | (pYES2) | |
| W303 tHMG1 ValS (evc) | W303 MATa PPGK1-tHMG7 PGAL1- | This study |
| | ValS (pYES2) | |
| W303 tHMG1 ValS pYES2-HPO-CPR | W303 MATa PPGK1-tHMG1 PGAL1- | This study |
| | ValS (pYES2 PGAL1-HPO-Flag | |
| | PGAL1-CPR-myc) | |
| W303 tHMG1 ValS Δice2 pYES2-HPO- | W303 MATa PPGK1-tHMG1 PGAL1- | This study |
| CPR | ValS Δice2 (pYES2 PGAL1-HPO- | |
| | Flag PGAL1-CPR-myc) | |
| W303 tHMG1 ValS PGAL1-ICE2-His6 | W303 MATa PPGK1-tHMG1 PGAL1- | This study |
| pYES2-HPO-CPR | ValS PGAL1-ICE2-His6 (pYES2 | |
| | PGAL1-HPO-Flag PGAL1-CPR-myc) | |
| W303 tHMG1 ValS PGAL1-ICE2-His6 | W303 MATa PPGK1-tHMG1 PGAL1- | This study |
| (evc) | ValS PGAL1-ICE2-His6 (pYES2) | |
| W303 tHMG1 ValS PICE2-ICE2-His6 | W303 MATa PPK1-tHMG1 PICE2- | This study |
| pYES2-HPO-CPR | ValS PGAL1-ICE2-His6 (pYES2 | |
| | PGAL1-HPO-Flag PGAL1-CPR-myc) | |
| W303 tHMG1 ValS PICE2-ICE2-His6 (evc) | W303 MATa PPGK1-tHMG1 PICE2-ValS | |
| | PGAL1-ICE2-His6 (pYES2) | |
| PpCBS7435 (wild type) | CBS7435 his4 | (Näätsaari et al., 2012) |
| Pp[HPO/CPR]ValS (HCV) | CBS7435 his4 ku70, | Wriessnegger et al., |
| | pPpHIS4[HPO/CPR], pPpB1- | manuscript in revision |
| | Zeocin[ValS] | |
| Pp[HPO/CPR]ValS/PpICE2 | CBS7435 his4 ku70, | This study |
| (HCV/PpICE2) | pPpHIS4[HPO/CPR], pPpB1- | |
| | Zeocin[ValS], pPpKan[PpICE2] | |
| Pp[PM17/AtCPR] | CBS7435 his4, pPpB1[PM17/AtCPR] | This study |
| Pp[PM17/AtCPR]PpICE2 | CBS7435 his4, | This study |
| | pPpB1[PM17/AtCPR], | |
| | pPpKan[PpICE2] | |
| Pp[2D6/hCPR] | CBS7435 his4, pPpB1 [2D6/hCPR] | This study |
| Pp[2D6/hCPR]PpICE2 | CBS7435 his4, pPpB1 [2D6/hCPR], | This study |
| | pPpKan[PpICE2] | |

TABLE 2
Primers used in this study (restriction sites underlined)

| Primer name | Primer sequence |
|---|---|
| Fw_CPR_HindIII | CCCAAGCTTCGAAACGCATATGACTTCTG |
| Rv_CPR-myc_BamHI | CGCGGATCCTTATTACAAATCCTCTTCAGAAATCAATTTTTGTTCCCAGACATCTCTCAAGTATCTACC |
| pYES2_AscI_fw | TTGGCGCGCCTTAATTAAACGGATTAGAAGCCGCCGAG |
| pYES2_BglII_rev | TTAGATCTGGCGCGCCCGATTCATTAATGCAGGGCC |
| Fw_HPOSc_HindIII | CCCAAGCTTCGAAACGCATATGCAATTCT |
| Rv_HPO-Flag_Sc | CGCGGATCCCTCGAGTTATTACTTATCGTCGTCATCCTTGTAATCCTCTCGGGAAGGTTGGTAA |
| BglII_Gal1_fw | TTGGCGCGCCTTAATTAAACGGATTAGAAGCCGCCGAG |
| AscI_CYC1_rev | TTAGATCTGGCGCGCCCGATTCATTAATGCAGGGCC |
| F1(trp1) | GTGAGTATACGTGATTAAGCACACAAAGGCAGCTTGGAGTCGGATCCCCGGGTTAATTAA |
| R1(trp1) | TGCACAAACAATACTTAAATAAATACTACTCAGTAATAACGAATTCGAGCTCGTTTAAAC |
| Fw(PPGK1_XmaI) | GTACCCGGGGATTATTTTAGATTCCTGACTTC |
| Rv(PPGK1_BamHI) | TATGGATCCTCTTGTTTTTATATTTGTTGTAAA |
| Fw(tHMG1_PstI) | GACCTGCAGGCACCCTGCAGACCAATTGGTGAAAACTG |
| Rv(tHMG1_HindIII) | TGCAAGCTTGGCCTAACACATGGTGCTGTTGTGCTT |
| Fw(ex_leu2_tHMG1) | AGCAATATATATATATATATTTCAAGGATATACCATTCTATGTAAAACGACGGCCAGT |
| Rv(ex_leu2_tHMG1) | TAAAGTTTATGTACAAATATCATAAAAAAAGAGAATCTTTCCGATTCATTAATGCAGC |
| F4(ICE2) | CGTAAAGTGTTGGTGGATCTTATAGTATTCGTGAAGAATTCGAGCTCGTTTAAAC |
| R2(ICE2) | CTGCATGAAGCTTTTGGACAAACTGGTCATTTTGAGATCCGGGTTTT |
| F1(ICE2) | GTGGCCGATCACGCTAAAGATTAGGCAACGCGGATCCCCGGGTTAATTAA |
| R1(ICE2) | GTATTTCACCTTCCTTTTTGTCTTCGCGTATTTGGCAAAGGAATTCGAGCTCGTTTAAAC |
| F1(ICE2start) | AGAGAGGTGCTGTTTGTGGCCGATCACGCTAAAGATTAGGCAACGATGACCAGTTTGTCCAAAAG |
| Fw(CDC73_qRT) | GAAAGGCGAGACATCCGATA |
| Rv(CDC73_qRT) | TTGTTTCCACCACAACTGGA |
| Fw(HPO_qRT) | ACATTGCGTTTTGCCCTTAC |
| Rv(HPO_qRT) | GCAACACTTCATCGCGTCTA |
| Fw(CPR_qRT) | GGTTGCTGGTTTCGTTGTCT |
| Rv(CPR_qRT) | ACCCAAGTCCAAGTCGTCAT |
| Fw(ICE2_qRT) | CGTCTGGCAGAAACATCAAA |
| Rv(ICE2_qRT) | AAGGACCCCCATAACACCTC |
| Fw(CPR_NotI) | CCTCACTAAAGGGCGGCCGCAACAAAATGACTTCTGCTTTGTACGC |
| Rv(CPRmyc_BglII) | TTAATTAAGAGCTCAGATCTTTATTACCAGACATCTCTCA |
| FwIce2Pp | CGGAATTCCGAAACGATGCCCAAGATACGCTCC |
| RvIce2Pp | ATAAGAATGCGGCCGCTTAGTGATGGTGATGGTGGTGTGTCTCCCAATTACTAGTCAAATTATC |

### Expression of Recombinant Proteins

For expression of recombinant proteins in S. cerevisiae, 300 mL baffled shake flasks containing 50 mL of synthetic defined growth media (6.7 g yeast nitrogen base w/o amino acids; 1 g drop-out powder consisting of equal amounts of adenine, lysine, tyrosine, histidine, leucine and tryptophane; 2% glucose) were inoculated to an OD600 of 0.1. Cell suspensions were shaken for 48 h at 130 rpm and 30° C. After centrifugation for 5 min at 1,062×g, cell pellets were resuspended in 50 mL of synthetic defined induction media containing 2% galactose and 0.7% raffinose instead of 2% glucose. Induction was carried out for 6 h at 130 rpm and 30° C.

Randomly chosen P. pastoris transformants were screened in 96-DWPs as previously described (Weis et al., 2004). In brief, cells were cultivated in 250 μL of BMGY medium for 24 h at 28° C., 320 rpm and 80% humidity. Induction was started by addition of 250 μL of BMMY (2% methanol). Methanol was added every 12 h to a final concentration of 1% until 48 h of induction. Cultivated transformants from DWPs were pinned onto plates containing up to 2 mg mL-1 Zeocin™ for screening of potential multicopy gene integration events of CYP/CPR. Transformants growing at high Zeocin concentration were picked for further Western Blot analyses for detection of Flag-tagged CYP and myc-tagged CPR proteins. Overexpression of PpICE2 was determined by Western Blot analysis using an antibody against the C-terminal 6×His-tag (His6).

### SOS-PAGE/Western Blotting (Fiji Quantification)

Five OD600 units of induced S. cerevisiae or P. pastoris cells were harvested and prepared for SDS-PAGE according to (Riezman et al., 1983). Ten μL of the resulting supernatants were separated under reducing conditions on a NuPAGE 4-12% Bis-Tris Gel (Invitrogen), and were then transferred onto nitrocellulose membrane (GE Healthcare, Chalfont St Giles, UK) in a wet blotting system. Protein loading was assessed by PonceauS staining of the membrane. Immunodetection was performed with commercially available rabbit anti-Flag, anti-myc or anti-polyHIS primary antibodies (Thermo Scientific, St. Leon-Rot, Germany), and with anti-rabbit (for myc) or anti-mouse (for Flag and HIS) secondary antibodies linked to horse radish peroxidase purchased from Sigma-Aldrich® (Steinheim, Germany). Western blots were developed by Super Signal West Pico chemiluminescent substrate (Thermo Scientific, St. Leon-Rot, Germany) and protein bands were detected using a G:BOX Chemi HR16 bioimaging system (Syngene, Cambridge, UK).

Quantification of Western blot signal intensities was done with Fiji as biological image analysis platform (Schindelin et al., 2012). Samples were loaded in triplicates and average signal intensities of HPO and CPR of the control strain were set to be 100%. The other signal intensities were normalized to those of the control strain.

### Quantitative RT-PCR

Three-hundred μL of galacatose-induced cell culture were harvested in a table top centrifuge and resuspended in RNAlater® in amounts recommended by the supplier (life technologies, Vienna, Austria). Purified RNA extracts were prepared using ZR Fungal/Bacterial RNA MiniPrep™ kit (ZymoResearch, Germany, Freiburg). RNA quality and concentrations were determined via NanoDrop ND2000 (Thermo Scientific, St. Leon-Rot, Germany) and ethidium bromide gel electrophoresis. qRT-PCR was performed in a 2-step procedure. The Revert Aid Premium First Strand cDNA Synthesis Kit from Thermo Scientific (St. Leon-Rot, Germany) was applied on 600 ng of total RNA as described in the manual for reverse transcription (RT). Reaction mixtures were incubated at 25° C. for 10 min, at 50° C. for 15 min and RT was terminated by heating at 85° C. for 5 min. For the quantitative PCR step, Maxima SYBR Green PCR MM (2×) from Thermo Scientific (St. Leon-Rot, Germany) was used. Then, 2.5 μL of cDNA template solution were mixed with 12.5 μL of Maxima SYBR Green qPCR Mix (2×, ROX added) followed by the addition of 1 μL of respective forward and reverse primers (qRT-labeled primers; Table 2). Primers had been constructed with the help of the Primer3 program comprising the recommended guidelines (frodawinitedu) (Koressaar and Remm, 2007; Untergasser et al., 2012). The reaction mixtures were filled up with nuclease free water to a final volume of 25 μL. A no-template control and a Reverse Transcriptase Minus (RT−) control were performed and tested negative for impurities or contaminant DNA. Following the two-step cycling protocol, mixtures were initially denatured at 95° C. for 10 min followed by 40 cycles of denaturation at 95° C. for 15 s and annealing/elongation at 60° C. for 60 s. Results were evaluated based on the ΔCₜ method via normalization of the observed values onto the house-keeping gene CDC73 (Reed et al., 1988).

### Resting Cells Assay for (+)-Valencene, (−)-Limonene and Bufuralol Conversion

### (+)-Valencene and (−)-Limonene Hydroxylation Assay

For bioconversions with S. cerevisiae, six-hundred OD₆₀₀ units of galactose-induced cells were harvested and resuspended in 2 mL of 50 mM KPᵢ buffer, pH 7.4. Cell suspensions were split into two equal aliquots in Pyrex tubes and 20 μL of 100 mM (+)-valencene or 300 mM (−)-limonene in DMSO as well as 1% of Triton® X-100 (Amresco, Solon, Ohio) were added, respectively. Pyrex tubes were only sealed loosely with screw caps to strike a balance between air supply for cells and substrate volatilization. Conversions were carried out for 16 h at 170 rpm and 30° C. Terpenoids were extracted with 500 μL of ethyl acetate using a VXR basic Vibraxe (IKA®, Staufen, Germany) at room temperature and maximum speed for 30 min. After phase separation by centrifugation for 15 min at 2,720×g, organic layers were verified via GC-MS and quantified via GC-FID.

Pre-selected Pichia pastoris transformants co-expressing PM17/CPR/PpIce2 were cultivated in shake flasks as described, but without addition of n-dodecane. After 48 h of induction, OD₆₀₀ of the cell cultures was determined and culture volumes corresponding to 300 OD₆₀₀ units were transferred to sterile PYREX® tubes. The cells were pelleted at 3220×g for 5 min in an Eppendorf 5810R centrifuge and the supernatants were discarded. Limonene substrate solution (300 mM (−)-limonene in DMSO, 1% Triton X-100) was added to the cell suspension to a final concentration of 6 mM. The reaction was carried out at 28° C. for 24 h at 170 rpm. Monoterpenoids were extracted with 500 μl ethyl acetate to be analyzed by GC-FID.

### Bufuralol 1′-Hydroxylation Assay

For S. cerevisiae, bufuralol conversion assays were conducted exactly as described by Geier et al. (2012).

For P. pastoris, transformants were cultivated in 96-DWPs as described. After 48 h of induction, OD₆₀₀ values of cultures in each well were determined followed by centrifugation of the DWP at 3,220×g for 5 min. The supernatant was discarded and the cell pellets were resuspended in 100 mM potassium phosphate buffer, pH 7.4. Prednisolone was added to the supernatants as an internal standard to a final concentration of 50 ng/μl. The reaction mix was measured by HPLC-MS according to the method described by (Geier et al., 2012).

### Bi-Phasic (+)-Valencene Synthesis and Bioconversion Assay

Adapting the protocol of Cankar et al. (2011), S. cerevisiae cells co-expressing HPO, CPR and ValS were cultivated in 50 mL synthetic defined growth media in 100 mL shake flasks, starting at an OD₆₀₀ of 0.1. After 24 h of shaking at 30° C. and 170 rpm, 1 mL of induction solution (20% galactose and 7% raffinose) and 1 mL of supplement solution, i.e. 0.167 g yeast nitrogen base and 25 mg dropout powder dissolved in sterile ddH₂O, were added to the cell suspension. Five mL of n-dodecane were added directly to the flasks to form second, organic phases. Cells were cultivated as before for 24 h, followed by a second addition of 1 mL each of induction and supplement solutions. Cells were induced for up to 72 h, before the n-dodecane phases were subjected to GC-MS and GC-FID analyses. Cultivation of P. pastoris strains expressing HPO/CPR and ValS in combination with PpIce2 was scaled up to 50 ml total volume in shake flasks. Therefore, 25 mL of pre-cultures in BMGY medium were grown for 48 h at 28° C., 140 rpm, followed by the addition of 25 ml of induction medium BMMY containing 2% methanol and n-dodecane at a final concentration of 10%. Induction was performed as for DWP cultivations. After 48 h of induction, the cultures were transferred to 50 mL plastic tubes, spun at 3220×g for 5 min and the organic layers were transferred into GC vials for GC-FID analysis.

### Cytochrome c Reductase Assay

CPR activity was estimated by its ability to reduce bovine heart cytochrome c (Phillips and Langdon, 1962).

P. pastoris and S. cerevisiae cell lysates were prepared by glass bead lysis according to the “Pichia Expression Kit” manual (life technologies, Vienna, Austria) with minor modifications. In brief, cell pellets were disrupted with equal volumes of glass beads in 1.5 mL reaction tubes with 200 μL of breaking buffer (50 mM NaH₂PO₄, pH 7.4, 1 mM PMSF, 1 mM EDTA, 5% glycerol), respectively. The cell suspensions were intermittently vortexed for 30 s and incubated on ice for 30 s, which was repeated for 10 cycles. After cell disruption, total cell lysates were centrifuged at 1500×g in a tabletop centrifuge to remove unbroken cells and cell debris. Protein amounts in cell lysates were quantified using the Bio-Rad (Bradford) protein assay with bovin serum albumin as a standard.

Twenty-five μL of the supernatants were mixed with 125 μL of 300 μM cytochrome c solutions and made up to a final volume of 650 μL with 50 mM Tris-HCl buffer, pH 7.5. Fifty μL of 50 mM KCN solution, pH 7.7, were added to yeast preparations to mask endogenous oxidase activities. The enzymatic reaction was started by the addition of 50 μL of 1.5 mM NADPH. The increase in absorption at 550 nm was recorded for 2 min using an UV/Vis DU800 spectrophotometer (Beckman Coulter, Brea, Calif.). Reductase activity was calculated based on a molar extinction coefficient of 21 mM⁻¹ cm⁻¹ for the reduced cytochrome c (Phillips and Langdon, 1962).

### Product Analysis by GC-MS and GC-FID

Terpenoid extracts in organic solvents, ie. ethyl acetate as well as n-dodecane, were initially analyzed by GC-MS for identification of compounds using reference standards and comparing the derived mass fragmentation spectra. A 30 m HP column (0.25 mm×0.25 μm) was used on a Hewlett-Packard 5890 Series II plus GC equipped with a 5972 series mass selective detector (MSD). Sample aliquots of 1 μL were injected in split mode (split ratio 20:1) at 220° C. injector and 280° C. detector temperatures with helium as carrier gas at constant flow rate of 32 cm/s. The oven temperature program was as follows: 70° C. for 1 min, 10° C./min ramp to 200° C., and 30° C./min ramp to 290° C. (2 min). MSD was operated in a mass range of 40-250 amu with 3.5 scans/s and at electron multiplier voltage of 1635 V.

GC-FID methods were developed for routine analyses of terpenoid samples. Therefore, we used a HP-5 column (crosslinked 5% Ph-Me Siloxane; 10 m×0.10 mm×0.10 μm) on a Hewlett-Packard 6890 GC equipped with a flame ionization detector (FID). Sample aliquots of 1 μL were injected in split mode (split ratio 30:1) at 250° C. injector temperature and 320° C. detector temperature with hydrogen as carrier gas and a flow rate set to 0.4 mL/min in constant flow mode (49 cm/s linear velocity). For analysis of (+)-valencene and its products, the oven temperature program was as follows: 100° C. for 1 min, 20° C./min ramp to 250° C., and 45° C./min ramp to 280° C. (0.5 min). The use of a high-speed/high-resolution column reduced the total run time to 9 min per sample, without any loss of chromatographic resolution (Wriessnegger et al. manuscript in revision).

For analysis of (−)-limonene and isopiperitenol, the following oven temperatures were used: 40° C. for 1 min, 4° C./min ramp to 90° C. and 30° C./min ramp to 280° C. (0.5 min). Total run time could be optimized to 22 min per sample.

### Electron Microscopy

S. cerevisiae strains were cultivated as described for expression of heterologous proteins. After 6 h of induction with galactose, cells were harvested at 2,500 rpm for 5 min in an Eppendorf 5810R centrifuge and the cell pellets were washed with distilled H₂O. The cells were fixed for 5 min in 1% aqueous KMnO₄ at room temperature, washed with distilled H₂O, and fixed in 1% aqueous KMnO₄ for 20 min. Fixed cells were washed four times in distilled water and incubated in 0.5% aqueous uranyl acetate over night at 4° C. The samples were dehydrated for 20 min, each, in a graded series of ethanol (50%, 70%, 90%, and 100%). Pure ethanol was then exchanged by propylene oxide, and specimen were gradually infiltrated with increasing concentrations (30%, 50%, 70% and 100%) of Agar 100 epoxy resin mixed with propylene oxide for a minimum of 3 h per step. Samples were embedded in pure, fresh Agar 100 epoxy resin and polymerized at 60° C. for 48 h. Ultra-thin sections of 80 nm were stained for 3 min with lead citrate and viewed with a Philips CM 10 transmission electron microscope.

### Example 1

### Screening for Effectors of CYP/CPR Function in Recombinant S. cerevisiae Strains

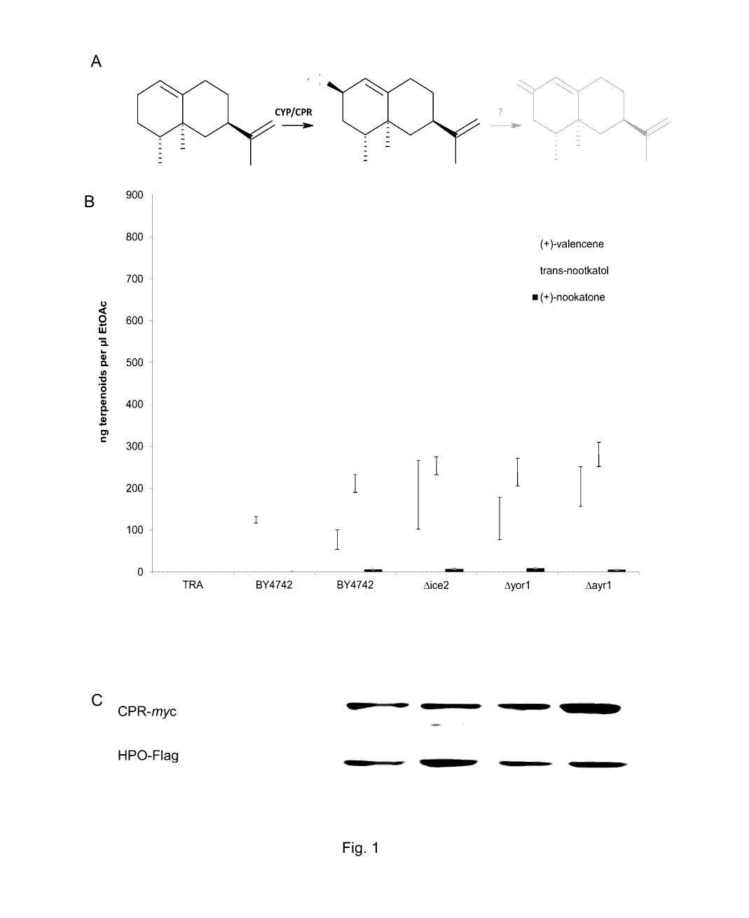

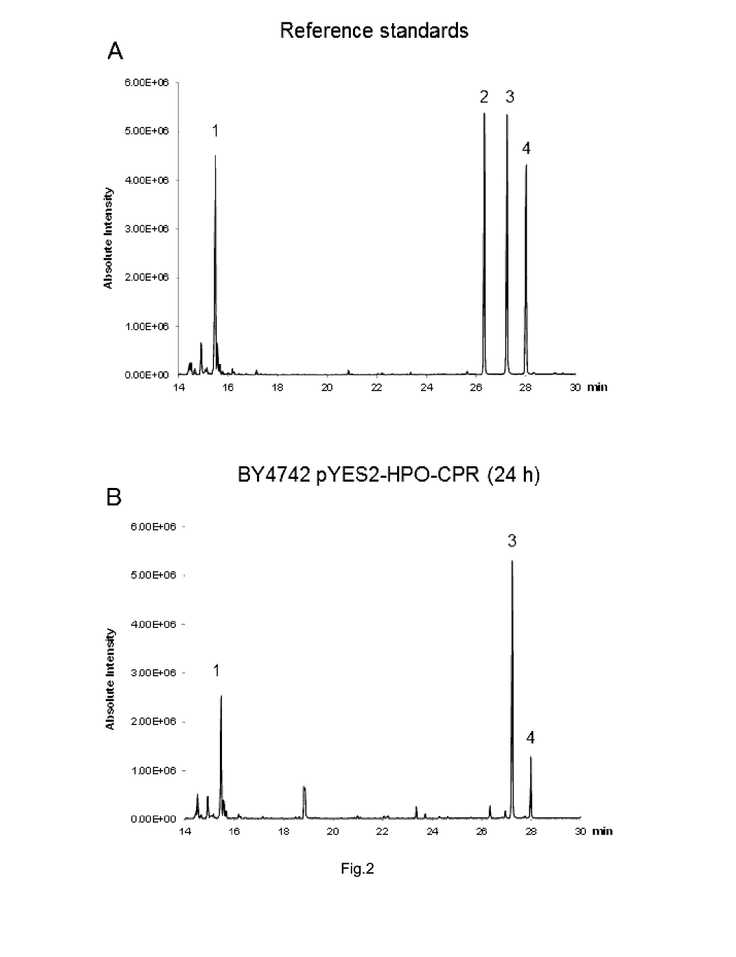

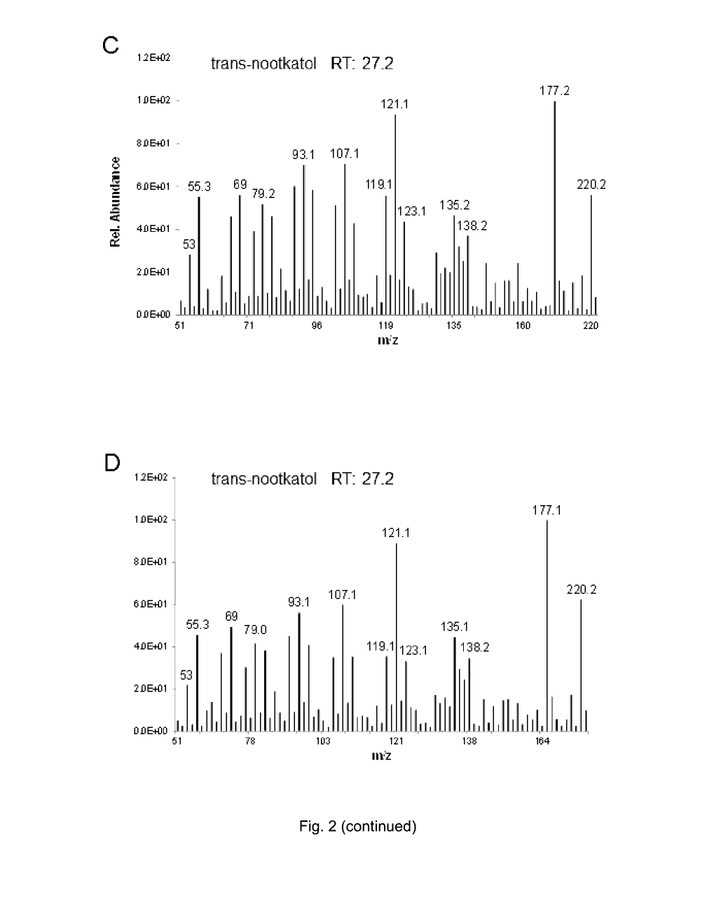

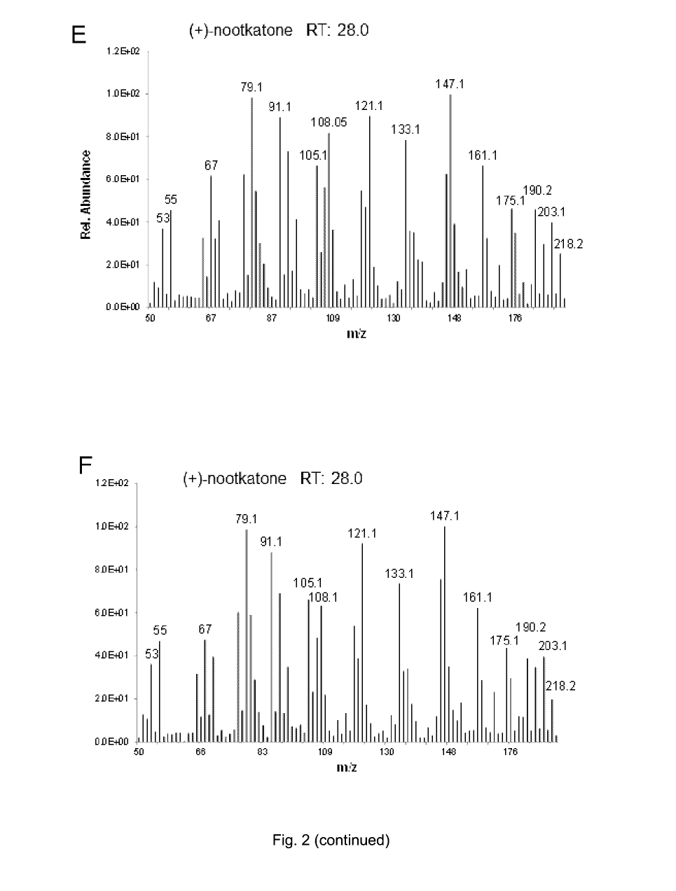

(+)-valencene is a side-product of orange juice production and of low commercial impact. However, there are numerous attempts to convert (+)-valencene into the attractive flavor and fragrance compound (+)-nootkatone by diverse biocatalytic approaches (reviewed by (Fraatz et al., 2009). As highly stereo- and regioselective catalysts, soluble and membrane-attached cytochrome P450 enzymes (CYPs) have been tested for performing this reaction (Cankar et al., 2011; Gavira et al., 2013; Girhard et al., 2009; Takahashi et al., 2007). There is solid data underscoring that CYPs do hydroxylate (+)-valencene to yield nootkatol (FIG. 1), but that oxidation of the latter compound to nootkatone is performed by endogenous activities of S. cerevisiae (Gavira et al., 2013) or Pichia pastoris (Wriessnegger et al., manuscript in revision). An important feature of CYPs is their requirement for cytochrome P450 reductase (CPR) activities regenerating the catalytically active CYPs. There are reports suggesting mutual stabilization of membrane-associated CYPs and CPRs necessitating balanced stoichiometry upon heterologous expression of such CYP/CPR pairs (reviewed by (Gonzalez and Korzekwa, 1995)(Murakami et al., 1986)(Geier et al., 2012)). Searching for protein effectors of CYP/CPR function, Hyoscyamus muticus premnaspirodiene oxygenase (HPO) variant V482I A484I (Takahashi et al., 2007) and Arabidopsis thaliana cytochrome P450 reductase (CPR) were functionally co-expressed from a multicopy vector (pYES2-HPO-CPR) in S. cerevisiae. Recombinant yeast strains performed (+)-valencene hydroxylation as expected from data in the literature (FIG. 1B). Compounds were verified by GC-MS (FIG. 2). Co-expression of HPO/CPR from a single vector enabled us to screen available yeast collections for effector proteins of HPO/CPR levels and activities. Selected single knockout mutants of the EUROSCARF collection were transformed with the pYES2-HPO-CPR plasmid and were tested for altered (+)-valencene conversion in resting cells assays. Most transformants showed conversion rates indistinguishable from the reference strain BY4742 pYES2-HPO-CPR (data not shown). A huge drawback of external (+)-valencene addition in resting cells assays was the high volatility of (+)-valencene in aqueous environment. After 20 h of conversion, only 20% of the initially added (+)-valencene was re-extractable from control cells harboring the empty pYES2 vector (FIG. 1B). Because side-products of (+)-valencene hydroxylation were not detected in GC-MS, the loss was ascribed to evaporation. In equivalent experiments, only 30% of the added trans-nootkatol evaporated within 20 h of shaking at 30° C. (data not shown). Aliquots of galactose-induced cells were routinely checked for HPO (53 kDa) and CPR (72 kDa) levels by Western blotting. Thereby, it was observed that CPR-myc appeared to be partially degraded in BY4742 Δice2, which was not observed in most other single knockout strains that were evaluated (FIG. 1C and data not shown). However, trans-nootkatol formation by HPO/CPR activity was not significantly different in BY4742 Δice2 as compared to the reference strain (FIG. 1B).

### Example 2

### Over-Expression of ICE2 in S. cerevisiae W303

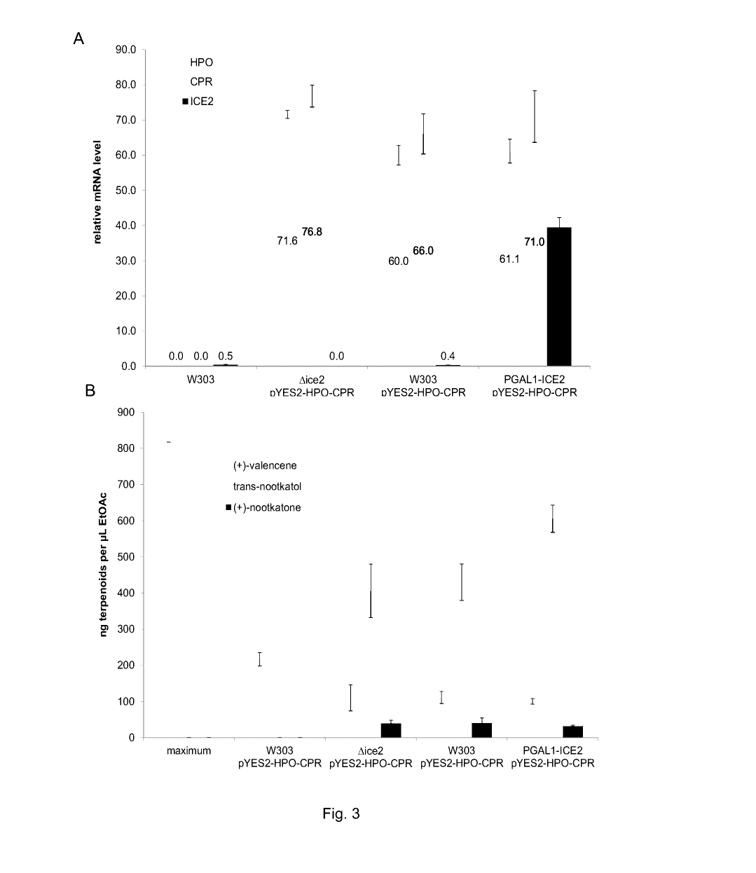

Among the various S. cerevisiae host strains used for CYP/CPR expression and CYP-mediated bioconversions, it has particularly been the W303 strain that turned out to be the most applicable host (Loeper et al., 1998; Pompon et al., 1996; Truan et al., 1993; Urban et al., 1994). Many of the standard lab strains of S. cerevisiae, but not the W303 strain, do carry a deletion in the HAP1 gene. Hap1p is a transcription factor responsible for regulation of genes governing intracellular heme abundance in response to changes in available oxygen levels (B. S. J. Davies and Jasper Rine, 2006; Ihrig et al., 2010). A Δhap1 genotype strain is supposedly less favorable for CYP expression and function as these are strongly dependent on optimal heme and oxygen supply. To test for the host strain effect, HPO/CPR-mediated resting cells conversion of (+)-valencene was performed in parallel in the BY4742 and the W303 yeast strains harboring pYES-HPO-CPR. As expected, (+)-valencene conversion levels were clearly higher in the W303 strain background—yielding 400 ng trans-nootkatol per μL of ethyl acetate (FIG. 3B)—than in the BY4742 strain background at 200 ng trans-nootkatol per μL of ethyl acetate (FIG. 1B). In both strain backgrounds, the knockout of Δice2 did not significantly influence the conversion of (+)-valencene to trans-nootkatol as compared to the corresponding reference strains. Since (+)-valencene conversion was substantially more efficient in the W303 strain background, further efforts were focussed on this host strain.

Triggered by the degradation products of CPR-myc observed in the Δice2 knockout strain (FIG. 1C), strains were created expressing ICE2 from the P_{GAL1} promoter inserted in front of the gene and used the engineered strains to be tested for HPO/CPR expression and (+)-valencene conversion, which were ideally controlled by the same promoter element. Interestingly, expressing ICE2 from P_{GAL1} resulted in 1.4-fold improved (+)-valencene conversion independent of the host strain background (FIG. 1B and FIG. 3B). The manipulations of Ice2 expression levels did not significantly affect cellular growth characteristics, as all strains grew to almost identical optical densities without and upon induction with galactose/raffinose (data not shown). To quantify expression of HPO, CPR and ICE2 on the mRNA level, quantitative real-time PCR was performed on all strains with modulated Ice2 expression. RNA was extracted after 6 h of galactose/raffinose induction and normalized against the housekeeping gene CDC73 in each sample. ICE2 appeared to be expressed at low levels in wild type cells (FIG. 3A), which is consistent with previous studies ascribing a total number of 606 molecules of Ice2p per cell growing exponentially in YPD (Ghaemmaghami et al., 2003). In the same study, 538 molecules of CDC73 were predicted under the same cultivation conditions, and to give comparison to another ER-membrane spanning protein, 24,800 molecules of Sec61p were found. Expressing ICE2 from the P_{GAL1} promoter resulted in 100-fold up-regulation of the gene as compared to the strains with ICE2 under the control of its own promoter (FIG. 3A). Interestingly, mRNA levels of HPO and CPR were largely independent of Ice2 modulation. Thus, the enhanced biohydroxylation of (+)-valencene observed for the W303 P_{GAL1}-ICE2 pYES2-HPO-CPR strain might indeed be elicited by posttranscriptional effects of elevated Ice2 expression on HPO/CPR protein levels and/or activity.

### Example 3

### In Vivo Bioconversion of (+)-Valencene in S. cerevisiae

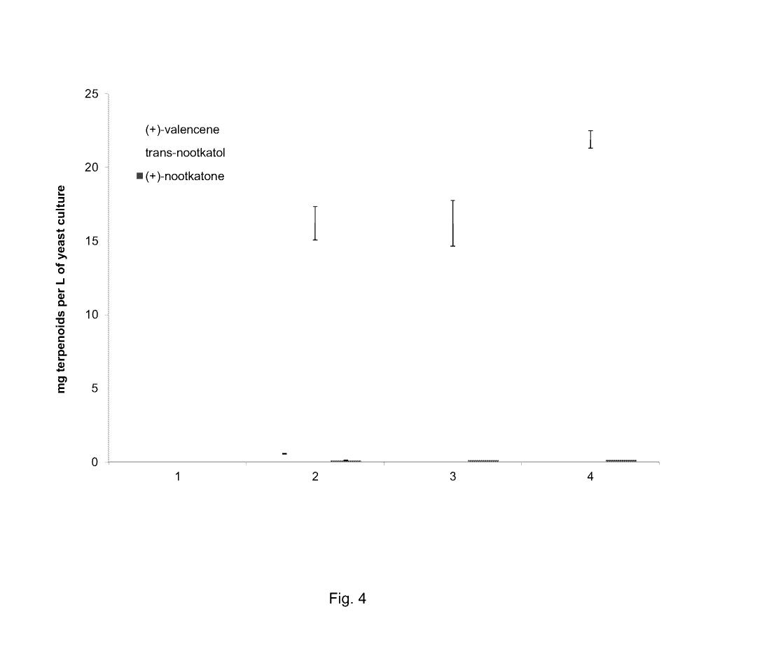

Assessing HPO/CPR-driven (+)-valencene hydroxylation in resting cells and quantifying the effect of P_{GAL1}-ICE2 expression thereon is subject to an important uncertainty factor, which is the availability of the (+)-valencene substrate. It is not only the evaporation of the terpene in aqueous environment that needs to be taken into account (FIG. 1B and FIG. 3B), but also diverse transport processes into and out of S. cerevisiae cells. To a pure hydrocarbon compound like (+)-valencene, the plasma membrane of yeast should not pose a stringent barrier for entry into the yeast cell. However, there are plenty of export mechanisms described for S. cerevisiae efficiently expelling hydrophobic compounds from the cell interior, e.g. diverse ABC transporters (Nishida et al., 2013)(reviewed by (Wawrzycka, 2011)), Pry proteins (Choudhary and Schneiter, 2012)(reviewed by (Jacquier and Schneiter, 2012)), etc. Thus, the intracellular (+)-valencene concentration, i.e. the substrate pool for HPO/CPR-mediated bioconversion, in resting cells assays is influenced by multiple factors that may be beyond the control of the experimenter. This situation obviously leads to the relatively high standard deviations in resting cells assays (FIG. 1B and FIG. 3B). Moreover, cell engineering, as for example over-expression of ICE2, might have an impact on the equilibrium of (+)-valencene transport across the yeast plasma membrane and, therefore, on intracellular availability of the compound. In order to avoid these uncertainties, strains were generated producing (+)-valencene intracellularly from FPP. Expression of sequence optimized valencene synthase (ValS) from C. nootkatensis in S. cerevisiae required co-expression of truncated Hmg1 in contrast to published work (Beekwilder et al., 2013). Strains expressing ValS without concomitant over-expression of tHMG1 grew extremely slowly, probably due to massive withdrawal of FPP from ergosterol biosynthesis (Parks et al., 1995; Song, 2003). Cells co-expressing ValS, tHMG1, HPO and CPR were initially cultivated according to the protocol described by Cankar et al. (2011), but cell growth was rather poor under these conditions. Conditions were adapted to pre-cultivating cells in 50 mL of glucose containing media for 24 h, followed by 24-72 h of induction via direct addition of galactose/raffinose to shake flasks. Overlaying the cell broth with 10% n-dodecane simultaneously with induction entrapped all terpenoids formed during bioconversions. Thereby, the W303 tHMG1 ValS pYES2-HPO-CPR strain produced roughly 20 mg of trans-nootkatol per liter of culture broth (FIG. 4). Remarkably, production of trans-nootkatol was increased by 50% upon expressing Ice2p-His₆ from the P_{GAL1} promoter resulting in a maximum productivity of 30 mg trans-nootkatol per liter of cell culture after 72 h of induction for the W303 tHMG1 ValS P_{GAL1}-ICE2 pYES2-HPO-CPR strain. Knocking out ICE2, on the other hand, did not influence the amount of terpenoids formed as compared to the reference strain (FIG. 4). Consequently, modulation of ICE2 expression did have equivalent effects upon HPO/CPR-mediated (+)-valencene hydroxylation in resting cells with externally added substrate (FIG. 3) and in cells producing the substrate in vivo (FIG. 4). These results underscore that the beneficial effect of Ice2 overexpression on HPO/CPR function was rather not due to altering bioavailability of the (+)-valencene substrate, but was much more likely due to altering HPO/CPR enzyme activity.

### Example 4

### ICE2 Overexpression Stabilizes CPR Levels and Activity

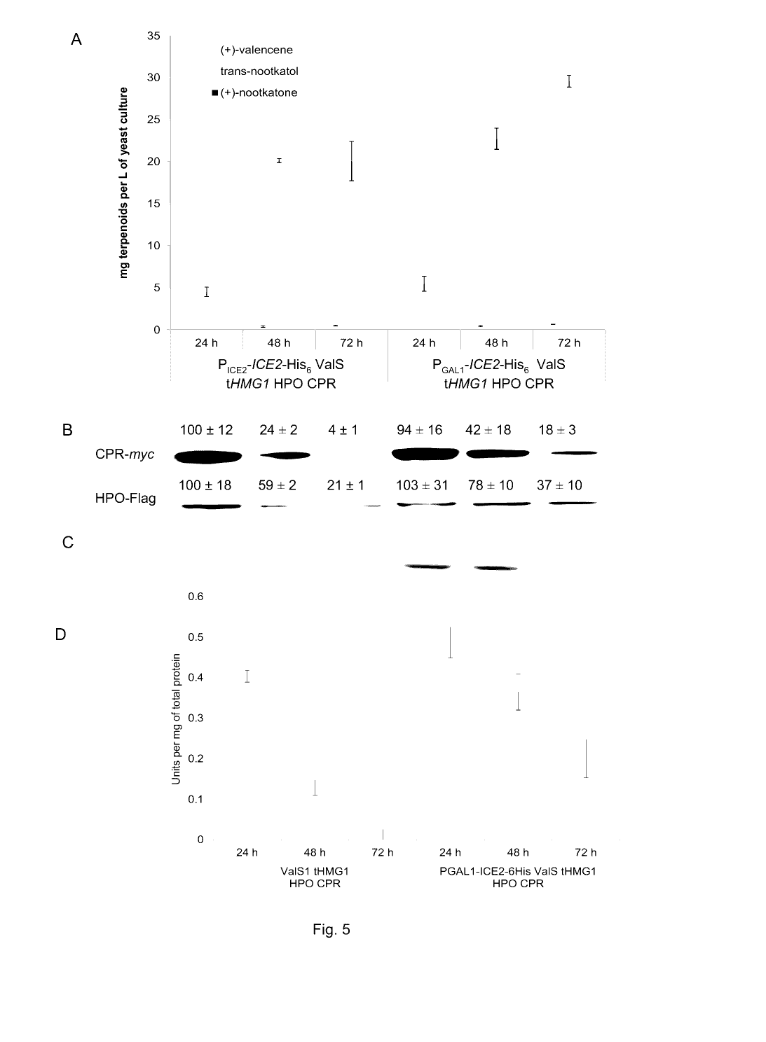

In an attempt to characterize the effects of ICE2 overexpression in more detail, strains were created that expressed Ice2p-His₆ either from its endogenous promoter or from P_{GAL1} and co-expressed HPO/CPR, ValS and tHMG1. These strains were subjected to time course bioconversion studies comparing HPO-Flag, CPR-myc and Ice2p-His₆ levels by western blotting besides quantifying terpenoid formation by GC (FIG. 5). Samples taken from the n-dodecane phase after 24 h, 48 h and 72 h of galactose/raffinose induction revealed that for the first 48 h of bioconversion there was only little difference in trans-nootkatol productivity 10%) between the strains that differed in the expression of Ice2p-His₆ (FIG. 5A). However, after 72 h of bioconversion the strain expressing Ice2p-His₆ from the galactose-inducible promoter formed 300 ng trans-nootkatol μL⁻¹ of n-dodecane (equivalent to 30 mg L⁻¹ of yeast culture), which was about 40% more than the 220 ng trans-nootkatol μL⁻¹ n-dodecane produced by the strain expressing Ice2p-His₆ from its native promoter. Western blot analysis showed that P_{GAL1}-driven ICE2 over-expression was not only detectable on the mRNA level (FIG. 3A), but also translated into higher protein levels at each time point (FIG. 5C). The Ice2-His₆ signals were hardly detectable under the control of the native promoter in 24 h samples and further decreased at later time points. However, Ice2p-His₆ levels were much higher with a slight decrease in 72 h samples when the protein was expressed from the galactose-inducible promoter. Most interestingly, HPO-Flag and, particularly, CPR-myc protein levels were clearly more stable over 72 h of bioconversion in the strain expressing higher levels of Ice2p-His₆ (FIG. 5B). It is reasonable to assume that PGAL1-driven Ice2p-His₆ expression enhanced (+)-valencene bioconversion by stabilizing CPR levels, because HPO action requires CPR for regeneration. Thus, the strain harboring elevated levels of Ice2p-His₆ and detectable amounts of CPR-myc will have further hydroxylated (+)-valencene until 72 h of conversion, in contrast to the strain that basically lacked CPR-myc protein at the last time point. HPO/CPR protein levels were quantified by densitometric scanning with the Fiji program (Schindelin et al., 2012). However, HPO/CPR protein levels might not be representative of HPO/CPR activity required for specific hydroxylation of (+)-valencene. Unfortunately, the active fraction of HPO could not be characterized by CO-difference spectroscopy (Omura, T., Sato, 1964) due to low signal intensities in whole cells and cell homogenates. Yet, the activities of cytochrome P450 reductase were quantified at different time points of bioconversion using the cytochrome c reductase assay. The evaluation of CPR activity is based on the reduction of oxidized cytochrome c and detecting reduced cytochrome at 550 nm (Phillips and Langdon, 1962). Reference strains without co-expression of HPO and CPR, ie. strains P_{ICE2}-ICE2-His₆ (evc) and P_{GAL1}-ICE2-His₆ (evc) were found to have background reductase activities of 0.030±0.004 and 0.042±0.007 U per mg of total protein for the W303 and P_{GAL1}-ICE2-6His strain, respectively (FIG. 5D). This was in good agreement with values reported for the W303 strain (Geier et al., 2012). Upon subtracting the background activity values, it was realized that CPR activity was virtually independent of ICE2 expression at 24 h of terpenoid bioconversion experiments, but strongly decreased at low Ice2p levels after 48 h of galactose/raffinose induction. While overexpression of Ice2 from the inducible promoter stabilized 75% of CPR activity over 72 h of assay time, CPR activity was not detectable in the strain expressing ICE2 from its native promoter (FIG. 5D). These values corresponded very well to the CPR-myc levels detected by western blotting (FIG. 5C). Thus, it is proposed that the positive effect of ICE2 overexpression on HPO/CPR-based (+)-valencene hydroxylation is mediated—at least to a large extent—by stabilization of CPR activity during in vivo terpenoid formation.

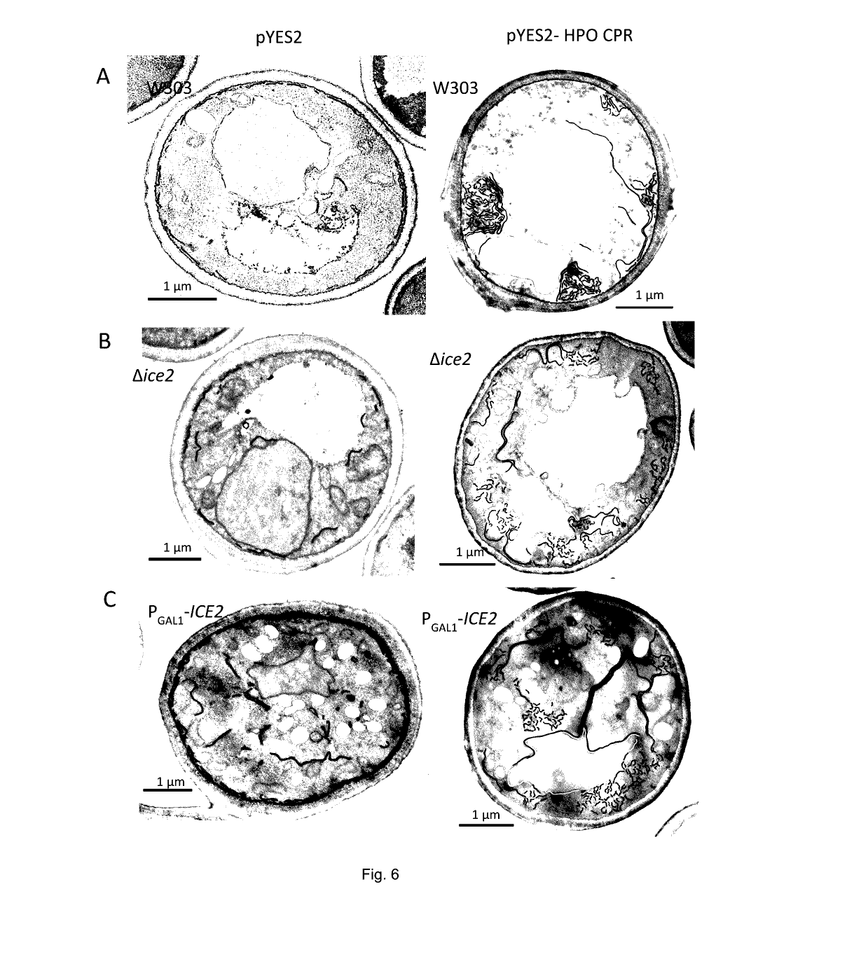

On the basis of previous studies reporting an altered cellular ER membrane distribution in S. cerevisiae cells disrupted for ice2 (Estrada De Martin, Du, Novick, & Ferro-Novick, 2005; Tavassoli et al., 2013), electron microscopy pictures of engineered strains were generated (FIG. 6). In general, overexpression of membrane proteins results in formation of staged ER-membranes, also called karmellae (Wright et al., 1988). The same effect was observed for W303 wild type, Δice2 and PGAL1-ICE2 strains overexpressing HPO and CPR (FIG. 6). As expected, knocking out of ice2 did slightly reduce amounts of peripheral ER membranes (FIG. 6B), although the effect was not as explicit as shown by Tavassoli et al., (2013) who presented electron microscopy pictures of the ice2 scs2 double knockout mutant. Interestingly, overexpression of ICE2 did lead to a significant increase in ER membranes close to the yeast plasma membrane (FIG. 6C).

### Example 5

### A General Effect of ICE2 Overexpression on CPR Stability

To test whether stabilization of CPR levels and/or activity is a general effect of ICE2 overexpression in S. cerevisiae and other yeast expression hosts, the analysis was extended to alternative CYP/CPR combinations in S. cerevisiae and P. pastoris as expression host.

As alternative CYP/CPR combinations we have chosen (−)-limonene-3-hydroxylase from Mentha piperita (PM17) with CPR as well as human cytochrome P450 2D6 (CYP2D6) with human reductase (hCPR). PM17 and CPR catalyze the conversion of the monoterpene (−)-limonene to (−)-trans-isopiperitenol (Karp et al., 1990; Lupien et al., 1995). CYP2D6 and hCPR act as main detoxifier of drugs in the human liver (Zhuge et al., 2004) and, therefore, hydroxylate the model substrate bufuralol to 1-hydroxybufuralol (Geier et al., 2012).

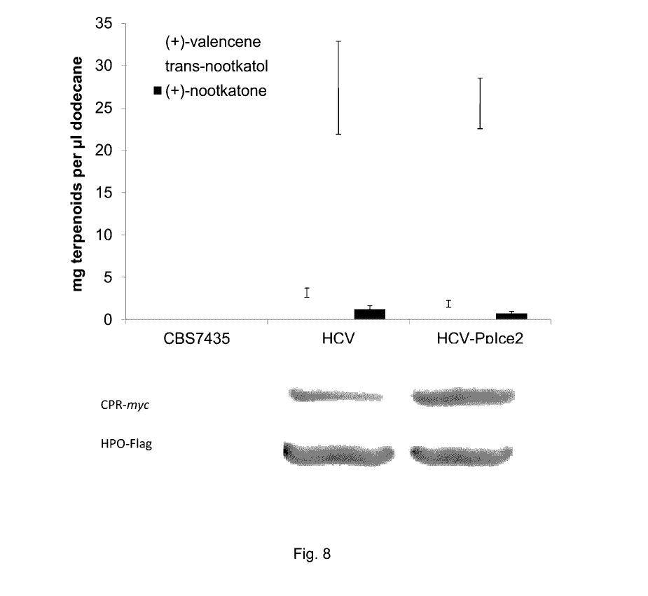

In S. cerevisiae, (−)-limonene conversion could not be increased by over-expression of ICE2 (FIG. 8A), however, conversion of bufuralol could be improved 1.5 fold (FIG. 8B). Western blot analysis did not reveal any strain dependent alterations of HPO and CPR levels (FIG. 8C). In contrast, analyses of CPR activities in Ice2 co-expression strains showed a massive effect on the stability within longer induction times (Table 3). After 6 h of induction, the reductases showed constantly higher activities if expressed in the Ice2 co expression strains. This effect was even more striking if cells were induced for up to 24 h, where reductase activities could be stabilized up to 4-fold in strains harboring Ice2p (Table 3).

TABLE 3
Reductase activities in U mg−1 of total protein of CYP/CPR pairs in S. cerevisiae
and P. pastoris. S. cerevisiae resting cells were induced for 6 h, which
is the time-point when resting cells assays where set up. Induction was prolonged to
24 h for analysis of reductase activities. P. pastoris strains were induced for
48 h and samples were drawn for cytochrome c reductase activity assays.
S. cerevisiae

| Plasmid | | | | pESC-URA PM17 | | PESC-URA CYP2D6 | |
|---|---|---|---|---|---|---|---|
| PGAL1- | | pYES2 HPO CPR | | CPR | | hCPR | |
| ICE2 | | − | + | − | + | − | + |
| 6 | h | 1.10 ± 0.09 | 1.55 ± 0.02 | 2.38 ± 0.23 | 3.26 ± 0.22 | 7.13 ± 0.03 | 8.37 ± 0.49 |
| 24 | h | 0.36 ± 0.02 | 1.06 ± 0.02 | 0.22 ± 0.01 | 0.82 ± 0.07 | 0.28 ± 0.001 | 0.92 ± 0.14 |

| P. pastoris | | | |
|---|---|---|---|
| Strain | HCV | PM17/CPR | CYP2D6/hCPR |

| PpICE2 | − | + | − | + | − | + |
|---|---|---|---|---|---|---|
| 48 h | 0.26 ± 0.02 | 0.37 ± 0.01 | 0.26 ± 0.01 | 0.34 ± 0.01 | 0.52 ± 0.00 | 0.69 ± 0.01 |

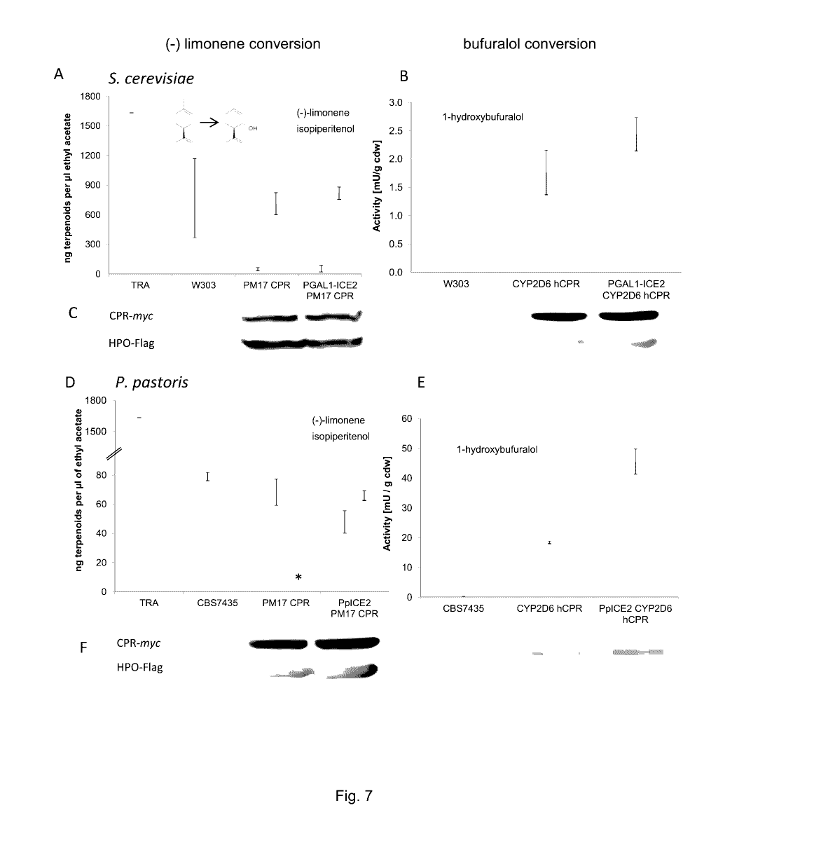

A P. pastoris strain producing (+)-valencene, trans-nootkatol and (+)-nootkatone was constructed by Wriessnegger et al. (manuscript in revision). Co-expression of PpICE2 could not improve yields of terpenoids (FIG. 7A) nor could an effect on CPR and HPO levels been detected (FIG. 7B).

Pichia pastoris had some troubles with converting (−)-limonene, although PM17 and CPR were expressed very well (FIGS. 8D and F). There are different studies where toxic effects of (−)-limonene have been described (Brennan et al., 2013; Liu et al., 2013). We assume that P. pastoris might have more efficient mechanisms to get rid of toxic substances such as monoterpenoids (own unpublished results) than S. cerevisiae. However, conversion of (−)-limonene was only detected, if PpICE2 was co-expressed (FIG. 8D). PpIce2p could improve conversion of bufuralol 2.5 fold (FIG. 8E). Interestingly, after 48 h of induction we observed a higher activity of reductases in all three P. pastoris co-expression systems (Table 3). With two different yeast expression hosts, S. cerevisiae and P. pastoris, we could show that over-expression of ICE2 improves conversion of two out of three substrates per strain. In all systems tested, a significant stabilization of cytochrome P450 reductases could be demonstrated proving the general validity of reductase stabilization by Ice2p.

### REFERENCES

Amberg, D. C., Burke, D. J., Strathern, J. N., 2006. Tetrad dissection. CSH protocols 2006, pdb.prot4181.
Asadollahi, M. A., Maury, J., Møller, K., Nielsen, K. F., Schalk, M., Clark, A., Nielsen, J., 2008. Production of plant sesquiterpenes in Saccharomyces cerevisiae: effect of ERG9 repression on sesquiterpene biosynthesis. Biotechnology and bioengineering 99, 666-77.
Asadollahi, M. A., Maury, J., Path, K. R., Schalk, M., Clark, A., Nielsen, J., 2009. Enhancing sesquiterpene production in Saccharomyces cerevisiae through in silico driven metabolic engineering. Metabolic Engineering 11, 328-334.
Ausubel, F. M., Brent, R., Kingston, R. E., Moore, D. D., Seidman, J. G., Smith, J. A., Struhl, K., Wiley, C. J., Allison, R. D., Bittner, M., Blackshaw, S., 2003. Current Protocols in Molecular Biology Current Protocols in Molecular Biology, Molecular Biology. John Wiley & Sons, Inc.
Beekwilder, J., Van Houwelingen, A., Cankar, K., Van Dijk, A. D. J., De Jong, R. M., Stoopen, G., Bouwmeester, H., Achkar, J., Sonke, T., Bosch, D., 2014. Valencene synthase from the heartwood of Nootka cypress (Callitropsis nootkatensis) for biotechnological production of valencene. Plant biotechnology journal 12, 174-182.
Bonneaud, N., Ozier-Kalogeropoulos, O., Li, G. Y., Labouesse, M., Minvielle-Sebastia, L., Lacroute, F., 1991. A family of low and high copy replicative, integrative and single-stranded S. cerevisiae/E. coli shuttle vectors. Yeast (Chichester, England) 7, 609-15.
Botstein, D., and Davis, R. W., 1982. The Molecular Biology of the Yeast Saccharomyces: Metabolism and Gene Expression. Cold Spring Harbor Laboratory Press, Cold Spring Harbor, N.Y. 607-636.
Brachmann, C. B., Davies, A., Cost, G. J., Caputo, E., Li, J, Hieter, P., Boeke, J. D., 1998. Designer deletion strains derived from Saccharomyces cerevisiae 5288C: a useful set of strains and plasmids for PCR-mediated gene disruption and other applications. Yeast (Chichester, England) 14, 115-32.
Brennan, T. C. R., Krömer, J. O., Nielsen, L. K., 2013. Physiological and transcriptional responses of Saccharomyces cerevisiae to d-limonene show changes to the cell wall but not to the plasma membrane. Applied and environmental microbiology 79, 3590-600.
Brown, M. S., Goldstein, J. L., 1980. Multivalent feedback regulation of HMG CoA reductase, a control mechanism coordinating isoprenoid synthesis and cell growth. Journal of lipid research 21, 505-17.
Cankar, K., Van Houwelingen, A., Bosch, D., Sonke, T., Bouwmeester, H., Beekwilder, J., 2011. A chicory cytochrome P450 mono-oxygenase CYP71AV8 for the oxidation of (+)-valencene. FEBS Letters 585, 178-182.
Cereghino, J. L., Cregg, J. M., 2000. Heterologous protein expression in the methylotrophic yeast Pichia pastoris, FEMS Microbiology Reviews.
Chambon, C., Ladeveze, V., Servouse, M., Blanchard, L., Javelot, C., Vladescu, B., Karst, F., 1991. Sterol pathway in yeast. Identification and properties of mutant strains defective in mevalonate diphosphate decarboxylase and farnesyl diphosphate synthetase. Lipids 26, 633-6.
Chang, M. C. Y., Keesling, Jay D, 2006. Production of isoprenoid pharmaceuticals by engineered microbes. Nature chemical biology 2,674-681.
Choudhary, V., Schneiter, R., 2012. Pathogen-Related Yeast (PRY) proteins and members of the CAP superfamily are secreted sterol-binding proteins. Proceedings of the National Academy of Sciences of the United States of America 109, 16882-7.
Danielson, P. B., 2002. The cytochrome P450 superfamily: biochemistry, evolution and drug metabolism in humans. Current drug metabolism 3, 561-97.
Davies, B. S. J., Rine, Jasper, 2006. A role for sterol levels in oxygen sensing in Saccharomyces cerevisiae. Genetics 174, 191-201.
Donald, K. A., Hampton, R Y, Fritz, I. B., 1997. Effects of overproduction of the catalytic domain of 3-hydroxy-3-methylglutaryl coenzyme A reductase on squalene synthesis in Saccharomyces cerevisiae. Applied and environmental microbiology 63, 3341-4.
Dong, M.-S., Lee, S.-B., Kim, H.-J., 2013. Co-expression of human cytochrome b5 increases expression of cytochrome P450 3A4 in Escherichia coli by stabilizing mRNA. Protein expression and purification 89, 44-50.
Estrada De Martin, P., Du, Y., Novick, P., Ferro-Novick, S., 2005. Ice2p is important for the distribution and structure of the cortical ER network in Saccharomyces cerevisiae. Journal of Cell Science 118, 65-77.
Farhi, M., Marhevka, E., Masci, T., Marcos, E., Eyal, Y., Ovadis, M., Abeliovich, H., Vainstein, A., 2011. Harnessing yeast subcellular compartments for the production of plant terpenoids. Metabolic engineering 13, 474-81.35
Fraatz, M. A., Berger, R. G., Zorn, H., 2009. Nootkatone-a biotechnological challenge. Applied microbiology and biotechnology 83, 35-41.
Gavira, C., Hofer, R., Lesot, A., Lambert, F., Zucca, J., Werck-Reichhart, Daniele, 2013. Challenges and pitfalls of P450-dependent (+)-valencene bioconversion by Saccharomyces cerevisiae. Metabolic engineering 18, 25-35.
Geier, M., Braun, A., Emmerstorfer, A., Pichler, H., Glieder, A., 2012. Production of human cytochrome P450 2D6 drug metabolites with recombinant microbes—a comparative study. Biotechnology journal 1-13.
Ghaemmaghami, S., Huh, W.-K., Bower, K., Howson, R. W., Belle, A., Dephoure, N., O'Shea, E. K., Weissman, J. S., 2003. Global analysis of protein expression in yeast. Nature 425, 737-41.
Gietz, R. D., Schiestl, R. H., 1995. Transforming yeast with DNA. Methods in Molecular and Cellular Biology 5, 255-269.
Girhard, M., Machida, K., Itoh, M., Schmid, R. D., Arisawa, A., Urlecher, V. B., 2009. Regioselective biooxidation of (+)-valencene by recombinant E. coli expressing CYP109B1 from Bacillus subtilis in a two-liquid-phase system. Microbial cell factories 8, 36.
Gonzalez, F. J., Korzekwa, K. R., 1995. Cytochromes P450 expression systems. Annual review of pharmacology and toxicology 35, 369-90.
Gu, J., Weng, Yan, Zhang, Q.-Y., Cui, H., Behr, M., Wu, L., Yang, W., Zhang, Li, Ding, X., 2003. Liver-specific deletion of the NADPH-cytochrome P450 reductase gene: impact on plasma cholesterol homeostasis and the function and regulation of microsomal cytochrome P450 and heme oxygenase. The Journal of Biological Chemistry 278, 25895-25901.
Hampton, R Y, Rine, J, 1994. Regulated degradation of HMG-CoA reductase, an integral membrane protein of the endoplasmic reticulum, in yeast. The Journal of cell biology 125, 299-312.
Hampton, R., Dimster-Denk, D., Rine, J, 1996. The biology of HMG-CoA reductase: the pros of contra-regulation. Trends in biochemical sciences 21, 140-5.
Henderson, C. J., Otto, D. M. E., Carrie, D., Magnuson, M. A., McLaren, A. W., Rosewell, I., Wolf, C. R., 2003. Inactivation of the hepatic cytochrome P450 system by conditional deletion of hepatic cytochrome P450 reductase. The Journal of Biological Chemistry 278, 13480-13486.
Ihrig, J., Hausmann, A., Hain, A., Richter, N., Hamza, I., Lill, R., MOhlenhoff, U., 2010. Iron regulation through the back door: iron-dependent metabolite levels contribute to transcriptional adaptation to iron deprivation in Saccharomyces cerevisiae. Eukaryotic cell 9, 460-71.
Jacquier, N., Schneiter, R., 2012. Mechanisms of sterol uptake and transport in yeast. The Journal of steroid biochemistry and molecular biology 129, 70-8. Karp, F., Mihaliak, C. A., Harris, J. L., Croteau, R., 1990. Monoterpene biosynthesis: specificity of the hydroxylations of (−)-limonene by enzyme preparations from peppermint (Menthe piperita), spearmint (Menthe spicata), and perilla (Perilla frutescens) leaves. Archives of biochemistry and biophysics 276, 219-26.
Kirby, J., Keesling, J. D., 2009. Biosynthesis of plant isoprenoids: perspectives for microbial engineering. Annual review of plant biology 60, 335-355.
Koressaar, T., Remm, M., 2007. Enhancements and modifications of primer design program Primer3. Bioinformatics (Oxford, England) 23, 1289-91.
Kwiatkowski, T. J., Zoghbi, H. Y., Ledbetter, S. A., Ellison, K. A., Chinault, A. C., 1990. Rapid identification of yeast artificial chromosome clones by matrix pooling and crude lysate PCR. Nucleic acids research 18, 7191-2.
Liu, J., Zhu, Y., Du, G., Zhou, J., Chen, Jian, 2013. Response of Saccharomyces cerevisiae to D-limonene-induced oxidative stress. Applied Microbiology and Biotechnology 97, 6467-75.
Loeper, J., Louerat-Oriou, B., Duport, C., Pompon, D., 1998. Yeast expressed cytochrome P450 2D6 (CYP2D6) exposed on the external face of plasma membrane is functionally competent. Molecular pharmacology 54, 8-13.
Longtine, M. S., McKenzie, A., Demarini, D. J., Shah, N. G., Wach, A., Brachat, A., Philippsen, P., Pringle, J. R., 1998. Additional modules for versatile and economical PCR-based gene deletion and modification in Saccharomyces cerevisiae. Yeast Chichester England 14, 953-61.
Lupien, S., Karp, F., Ponnamperuma, K., Wildung, M., Croteau, R., 1995. Cytochrome P450 limonene hydroxylases of Mentha species. Drug metabolism and drug interactions 12, 245-60.
Majetich G., Behnke M., H. K., 1985. A Stereoselective Synthesis of (+/−)-Nootkatone and (+/−)-Valencene via an Intramolecular Sakurai Reaction. J. Org. Chem 50, 3615-3618.36
Murakami, H., Yabusaki, Y., Ohkawa, H., 1986. Expression of rat NADPH-cytochrome P-450 reductase cDNA in Saccharomyces cerevisiae. DNA (Mary Ann Liebert, Inc.) 5, 1-10.
Murataliev, M. B., Guzov, V. M., Walker, F. A., Feyereisen, R., 2008. P450 reductase and cytochrome b5 interactions with cytochrome P450: effects on house fly CYP6A1 catalysis. Insect biochemistry and molecular biology 38, 1008-15.
Näätsaari, L., Mistlberger, B., Ruth, C., Hajek, T., Hartner, F. S., Glieder, A., 2012. Deletion of the Pichia pastoris KU70 homologue facilitates platform strain generation for gene expression and synthetic biology. PloS one 7, e39720.
Nishida, N., Jing, D., Kuroda, K., Ueda, M. Activation of signaling pathways related to cell wall integrity and multidrug resistance by organic solvent in Saccharomyces cerevisiae. Current Genetics, Epub ahead of print Dec. 31, 2013
Nishida, N., Ozato, N., Matsui, K., Kuroda, K., Ueda, M., 2013b. ABC transporters and cell wall proteins involved in organic solvent tolerance in Saccharomyces cerevisiae. Journal of biotechnology 165, 145-52.
North, M., Steffen, J., Loguinov, A. V, Zimmerman, G. R., Vulpe, C. D., Eide, D. J., 2012. Genome-Wide Functional Profiling Identifies Genes and Processes Important for Zinc-Limited Growth of Saccharomyces cerevisiae. PLoS Genetics 8, e1002699.
Omura, T., 2010. Structural diversity of cytochrome P450 enzyme system. Journal of Biochemistry 147, 297-306.
Omura, T., Sato, R., 1964. The carbon monoxide-binding pigment of liver microsomes. I. Evidence for its hemoprotein nature. The Journal of biological chemistry 239, 2370-8.
Parks, L. W., Smith, S. J., Crowley, J. H., 1995. Biochemical and physiological effects of sterol alterations in yeast-a review. Lipids 30, 227-30.
Phillips, A. H., Langdon, R. G., 1962. Hepatic triphosphopyridine nucleotide-cytochrome c reductase: isolation, characterization, and kinetic studies. The Journal of biological chemistry 237, 2652-60.
Polakowski, T., Stahl, U., Lang, C., 1998. Overexpression of a cytosolic hydroxymethylglutaryl-CoA reductase leads to squalene accumulation in yeast. Applied Microbiology and Biotechnology 49, 66-71.
Pompon, D., Louerat, B., Bronine, A., Urban, P., 1996. Yeast expression of animal and plant P450s in optimized redox environments. Methods in enzymology 272, 51-64.
Reed, S I., Ferguson, J., Jahng, K. Y., 1988. Isolation and characterization of two genes encoding yeast mating pheromone signaling elements: CDC72 and CDC73. Cold Spring Harbor symposia on quantitative biology 53 Pt 2, 621-7.
Riezman, H, Hase, T., Van Loon, A. P., Grivell, L. A., Suda, K., Schatz, G., 1983. Import of proteins into mitochondria: a 70 kilodalton outer membrane protein with a large carboxy-terminal deletion is still transported to the outer membrane. The EMBO journal 2, 2161-8.
Ro, D.-K., Paradise, E. M., OueIlet, M., Fisher, K. J., Newman, K. L., Ndungu, J. M., Ho, K. A., Eachus, R. A., Ham, T. S., Kirby, J., Chang, M. C. Y., Withers, S T., Shiba, Y., Sarpong, R., Keesling, J. D., 2006. Production of the antimalarial drug precursor artemisinic acid in engineered yeast. Nature 440, 940-943.
Salvador, J. A. R., Clark, J. H., 2002. The allylic oxidation of unsaturated steroids by tert-butyl hydroperoxide using surface functionalised silica supported metal catalysts. Green Chemistry 4, 352-356.
Scalcinati, G., Knuf, C., Partow, S., Chen, Y., Maury, J., Schalk, M., Daviet, L., Nielsen, J., Siewers, V., 2012. Dynamic control of gene expression in Saccharomyces cerevisiae engineered for the production of plant sesquitepene α-santalene in a fed-batch mode. Metabolic engineering 14, 91-103.
Schindelin, J., Arganda-Carreras, I., Frise, E., Kaynig, V., Longair, M., Pietzsch, T., Preibisch, S., Rueden, C., Saalfeld, S., Schmid, B., Tinevez, J.-Y., White, D. J., Hartenstein, V., Eliceiri, K., Tomancak, P., Cardona, A., 2012. Fiji: an open-source platform for biological-image analysis. Nature methods 9, 676-82.
Song, L., 2003. Detection of farnesyl diphosphate accumulation in yeast ERG9 mutants. Analytical biochemistry 317, 180-5.
Takahashi, S., Yeo, Y.-S., Zhao, Yuxin, O'Maille, P. E., Greenhagen, B. T., Noel, J. P., Coates, R. M., Chappell, J., 2007. Functional characterization of premnaspirodiene oxygenase, a cytochrome P450 catalyzing regio- and stereo-specific hydroxylations of diverse sesquiterpene substrates. The Journal of Biological Chemistry 282, 31744-31754.
Truan, G., Cullin, C., Reisdorf, P., Urban, P., Pompon, D., 1993. Enhanced in vivo monooxygenase activities of mammalian P450s in engineered yeast cells producing high levels of NADPH-P450 reductase and human cytochrome b5. Gene 125, 49-55.
Untergasser, A., Cutcutache, I., Koressaar, T., Ye, J., Faircloth, B. C., Remm, M., Rozen, S. G., 2012. Primer3-new capabilities and interfaces. Nucleic acids research 40, e115.

1. A recombinant host cell which is capable of expressing a cytochrome P450 enzyme (CYP) and a cytochrome P450 reductase (CPR) and which is capable of overexpressing a Ice2p comprising the amino acid sequence set out in SEQ ID NO: 2 or a sequence having at least 50% sequence identity thereto.

2. A recombinant host cell according to claim 1, wherein the CYP is premnaspirodiene oxygenase CYP71D55 from Hyoscymaus muticus (HPO), optionally in which the mutations V482I and A484I have been made, (−)-limonene-3-hydroxylase from Mentha piperita (PM17) or human cytochrome P450 2D6 (CYP2D6).

3. A recombinant host cell according to claim 1, wherein the CRP is a cytochrome P450 reductase from A. thaliana or a human reductase.

4. A recombinant host cell according to claim 1 which is capable of production of a compound of interest.

5. A recombinant host cell according to claim 4, wherein the compound of interest is a sterol, a steviol glycoside, trans-nootkatol or nootkatone.

6. A recombinant host cell according to claim 1 wherein the host cell is a eukaryotic or a prokaryotic cell.

7. A recombinant host cell according to claim 6 which is a eukaryotic cell, optionally a fungal cell, optionally a yeast cell selected from the group consisting of Candida, Hansenula, Issatchenkia, Kluyveromyces, Pichia, Saccharomyces, Schizosaccharomyces, or Yarrowia strains, or a filamentous fungal cell selected from the group consisting of filamentous fungal cells belong to a species of Acremonium, Aspergillus, Chrysosporium, Myceliophthora, Penicillium, Talaromyces, Rasamsonia, Thielavia, Fusarium or Trichoderma.

8. A recombinant host cell according to claim 6 which is a prokaryotic cell, optionally a bacterial cell, optionally a bacterial cell belonging to the genus Bacillus, Escherichia, Pseudomonas, Lactobacillus.

9. A method for production of a compound of interest in a recombinant host cell, which method comprises:
providing a recombinant host cell according to claim 1 which is capable of expressing a compound of interest;
cultivating the recombinant host cell under conditions suitable for production of the compound of interest; and, optionally
recovering the compound of interest.

10. A method for production of a compound of interest in a biocatalytic reaction, which method comprises:
providing a recombinant host cell according to claim 1 which is capable of producing a CYP and CPR of interest;
cultivating the recombinant host cell under conditions suitable for production of CYP and CPR;
converting a suitable substrate to a compound of interest by contacting the substrate with the recombinant host cell or a biocatalyst formulation derived therefrom; and, optionally recovering the compound of interest.

11. A method according to claim 9 for conversion of valencene to trans-nootkatone and/or in conversion of trans-nootkatone to nootkatone

12. A product that is used to stabilize expression of a cytochrome P450 reductase in a recombinant host cell comprising Ice2p.


SEQUENCE LISTING (35 sequences)

SEQ ID NO: 1 - DNA, 1476 bp, Saccharomyces cerevisiae
  CDS (1)..(1476)
atgaccagtttgtccaaaagcttcatgcagagtggacgaatctgcgcagcatgtttctatctgttattcacactactttcaattccaatctcgtttaaagttggtggtttggaatgcgggctttccttcacggtgacactgttcactttatatttcataactacgactcttaacgtgttggcaagacgacatggaggaagactatacattttttttaccaactgtctgtattactcacaacattttatcattgcatctttgctatacctgtttttgtctggattttctaatgatgagttgggaaacgttctgaaaaataaatataatgagtcggagtcgttcctggaagctttgaaaaatagcttgaattccaatcaaattaactacgtcttatattattactactatcgatttgttgtacaaccgtggcaattcgtgcttaccaagtccacacctttttttactctatcggaaggttttttcactattttagccattcaggccgtcggggaaactaatagatggttatcaaatgacttgaattcaaacacgtggattatttcctcattgttaacctccggaggtgtgattaccgcatcgctgtactatttgtatcggatttatgtcacccccatatggccgttatccatccaaacggcgtccttattaggacttgttttgtctatggtatgtggactggggttgtatggtattgtgagtcaaaaaggatccgtcatagagagctctttattttttgcgtatattgttcgttgtatttatgaaatttcccccaaattagctactaccgcgactgatgaaattttaaatttgttcaaagacgtctggcagaaacatcaaagaaatctgcccacagctgacaatcttttgtgctactttcataatgtcatattgaaaaatgcagaggtgttatgggggtcctttattcctagaggaagaaagaaaaccggtgattttcatgataaactcattagcattctatcattcgaaaaagtatccttgatatctaaaccattttggaaatttttcaagaatttcacctttagtgttccgctatccattaatgaattttgtcaagttacaattaagatggcaagcgaatcagtttccccagctatagtaatcaatttatgctttagagttctgatgttttactcggcaacgaggattattccagcattacaaagaaaaaatgacaaacagttgcgcaagagtcgcaggatcatgaagggattgtattggtacagtccttgcatattaattgctatgtatactcacctgattttacaatattcaggtgagctaaagaaagacctgtgcatatggggttgcagtgaaaagtggtttggcgtagatcaaccagaaattatagtagattcatggggattttggaactggtgcaacattttctgtactattttggtatacgctacagaattaataggttctggtagttga

SEQ ID NO: 2 - protein, 491 aa, Saccharomyces cerevisiae
MTSLSKSFMQSGRICAACFYLLFTLLSIPISFKVGGLECGLSFTVTLFTLYFITTTLNVLARRHGGRLYIFFTNCLYYSQHFIIASLLYLFLSGFSNDELGNVLKNKYNESESFLEALKNSLNSNQINYVLYYYYYRFVVQPWQFVLTKSTPFFTLSEGFFTILAIQAVGETNRWLSNDLNSNTWIISSLLTSGGVITASLYYLYRIYVTPIWPLSIQTASLLGLVLSMVCGLGLYGIVSQKGSVIESSLFFAYIVRCIYEISPKLATTATDEILNLFKDVWQKHQRNLPTADNLLCYFHNVILKNAEVLWGSFIPRGRKKTGDFHDKLISILSFEKVSLISKPFWKFFKNFTFSVPLSINEFCQVTIKMASESVSPAIVINLCFRVLMFYSATRIIPALQRKNDKQLRKSRRIMKGLYWYSPCILIAMYTHLILQYSGELKKDLCIWGCSEKWFGVDQPEIIVDSWGFWNWCNIFCTILVYATELIGSGS

SEQ ID NO: 3 - DNA, 29 bp, Artificial sequence
  Primer Fw_CPR_HindIII
cccaagcttcgaaacgcatatgacttctg

SEQ ID NO: 4 - DNA, 69 bp, Artificial sequence
  Primer Rv_CPR-myc_BamHI
cgcggatccttattacaaatcctcttcagaaatcaatttttgttcccagacatctctcaagtatctacc

SEQ ID NO: 5 - DNA, 38 bp, Artificial sequence
  Primer pYES2_AscI_fw
ttggcgcgccttaattaaacggattagaagccgccgag

SEQ ID NO: 6 - DNA, 36 bp, Artificial sequence
  Primer pYES2_BglII_rev
ttagatctggcgcgcccgattcattaatgcagggcc

SEQ ID NO: 7 - DNA, 29 bp, Artificial sequence
  Primer Fw_HPOSc_HindIII
cccaagcttcgaaacgcatatgcaattct

SEQ ID NO: 8 - DNA, 64 bp, Artificial sequence
  Primer Rv_HPO-Flag_Sc
cgcggatccctcgagttattacttatcgtcgtcatccttgtaatcctctcgggaaggttggtaa

SEQ ID NO: 9 - DNA, 38 bp, Artificial sequence; identical to SEQ ID NO: 5
  Primer BglII_Gal1_fw

SEQ ID NO: 10 - DNA, 36 bp, Artificial sequence; identical to SEQ ID NO: 6
  Primer AscI_CYC1_rev

SEQ ID NO: 11 - DNA, 60 bp, Artificial sequence
  Primer F1(trp1)
gtgagtatacgtgattaagcacacaaaggcagcttggagtcggatccccgggttaattaa

SEQ ID NO: 12 - DNA, 60 bp, Artificial sequence
  Primer R1(trp1)
tgcacaaacaatacttaaataaatactactcagtaataacgaattcgagctcgtttaaac

SEQ ID NO: 13 - DNA, 32 bp, Artificial sequence
  Primer Fw(PPGK1_XmaI)
gtacccggggattattttagattcctgacttc

SEQ ID NO: 14 - DNA, 33 bp, Artificial sequence
  PrimerRv(PPGK1_BamHI)
tatggatcctcttgtttttatatttgttgtaaa

SEQ ID NO: 15 - DNA, 38 bp, Artificial sequence
  Primer Fw(tHMG1_PstI)
gacctgcaggcaccctgcagaccaattggtgaaaactg

SEQ ID NO: 16 - DNA, 36 bp, Artificial sequence
  Primer Rv(tHMG1_ HindIII)
tgcaagcttggcctaacacatggtgctgttgtgctt

SEQ ID NO: 17 - DNA, 58 bp, Artificial sequence
  Primer Fw(ex_leu2_tHMG1)
agcaatatatatatatatatttcaaggatataccattctatgtaaaacgacggccagt

SEQ ID NO: 18 - DNA, 58 bp, Artificial sequence
  Primer Rv(ex_leu2_tHMG1)
taaagtttatgtacaaatatcataaaaaaagagaatctttccgattcattaatgcagc

SEQ ID NO: 19 - DNA, 55 bp, Artificial sequence
  Primer F4 (ICE2)
cgtaaagtgttggtggatcttatagtattcgtgaagaattcgagctcgtttaaac

SEQ ID NO: 20 - DNA, 47 bp, Artificial sequence
  Primer R2 (ICE2)
ctgcatgaagcttttggacaaactggtcattttgagatccgggtttt

SEQ ID NO: 21 - DNA, 50 bp, Artificial sequence
  Primer F1 (ICE2)
gtggccgatcacgctaaagattaggcaacgcggatccccgggttaattaa

SEQ ID NO: 22 - DNA, 60 bp, Artificial sequence
  Primer R1 (ICE2)
gtatttcaccttcctttttgtcttcgcgtatttggcaaaggaattcgagctcgtttaaac

SEQ ID NO: 23 - DNA, 65 bp, Artificial sequence
  Primer F1 (ICE2start)
agagaggtgctgtttgtggccgatcacgctaaagattaggcaacgatgaccagtttgtccaaaag

SEQ ID NO: 24 - DNA, 20 bp, Artificial sequence
  Primer Fw(CDC73_qRT)
gaaaggcgagacatccgata

SEQ ID NO: 25 - DNA, 20 bp, Artificial sequence
  Primer Rv(CDC73_qRT)
ttgtttccaccacaactgga

SEQ ID NO: 26 - DNA, 20 bp, Artificial sequence
  Primer Fw(HPO_qRT)
acattgcgttttgcccttac

SEQ ID NO: 27 - DNA, 20 bp, Artificial sequence
  Primer Rv(HPO_qRT)
gcaacacttcatcgcgtcta

SEQ ID NO: 28 - DNA, 20 bp, Artificial sequence
  Primer Fw(CPR_qRT)
ggttgctggtttcgttgtct

SEQ ID NO: 29 - DNA, 20 bp, Artificial sequence
  Primer Rv(CPR_qRT)
acccaagtccaagtcgtcat

SEQ ID NO: 30 - DNA, 20 bp, Artificial sequence
  Primer Fw(ICE2_qRT)
cgtctggcagaaacatcaaa

SEQ ID NO: 31 - DNA, 20 bp, Artificial sequence
  Primer Rv(ICE2_qRT)
aaggacccccataacacctc

SEQ ID NO: 32 - DNA, 46 bp, Artificial sequence
  Primer Fw(CPR_NotI)
cctcactaaagggcggccgcaacaaaatgacttctgctttgtacgc

SEQ ID NO: 33 - DNA, 40 bp, Artificial sequence
  Primer Rv(CPRmyc_BglII)
ttaattaagagctcagatctttattaccagacatctctca

SEQ ID NO: 34 - DNA, 33 bp, Artificial sequence
  Primer FwIce2Pp
cggaattccgaaacgatgcccaagatacgctcc

SEQ ID NO: 35 - DNA, 64 bp, Artificial sequence
  Primer RvIce2Pp
ataagaatgcggccgcttagtgatggtgatggtggtgtgtctcccaattactagtcaaattatc